SEQUENCE LISTING (19 sequences)

SEQ ID NO: 1 - protein, 69 aa, artificial sequence
  neoantigenic construct
PSFQEFVDWENVSPELNSTDQVVRHLLGRLAAIVGKQVVLGRKVVVVRHWNDLAVIPAGVVHNWDFEPR

SEQ ID NO: 2 - protein, 69 aa, artificial sequence
  neoantigenic construct
VELCPGNKYEMRRHGTTHSLVVVRDEVALVEGVQSLGFTYLRLKDVVRKAFLHWYTGEAMDEMEFTEAE

SEQ ID NO: 3 - protein, 68 aa, artificial sequence
  neoantigenic construct
EAPRMPEAAPRVAPAPAAPTPVVRQSQHMTEVVRHCPHHERCSDSVVRCATYVNVNIRNIDKIYVRTG

SEQ ID NO: 4 - protein, 72 aa, artificial sequece
  neoantigenic construct
AQTYTLDVLERCPHRPILQAGLVVRSTRDPLSEITKQEKDFLWSHRVVRLVEADEAGSVCAGILSYGVGFGS

SEQ ID NO: 5 - protein, 72 aa, artificial sequence
  neoantigenic construct
AKAISTRDPLSKITEQEKDFLWVVRPYNYLSTDVGFCTLVCPLHNQVVRRQTYTLDVLECSPHRPILQAGGS

SEQ ID NO: 6 - protein, 72 aa, artificial sequence
  neoantigenic construct
AALLALWLCCATPAHALQCRDGVVRVKEGWLHKRGKYIKTWRPRYFVVREYFMKQMNDARHGGWTTKMDWGS

SEQ ID NO: 7 - protein, 72 aa, artificial sequence
  neoantigenic construct
ATEYKLVVVGAVGVGKSALTIQVVREEELVEADEACSVYAGILSYGVVRCACPGRDRRTKEENLRKKGEPGS

SEQ ID NO: 8 - protein, 72 aa, artificial sequence
  neoantigenic construct
ATEYKLVVVGADGVGKSALTIQVVRFEVRVCACPGTDRRTEEENLRVVRCLLDILDTAGREEYSAMRDQYGS

SEQ ID NO: 9 - protein, 50 aa, artificial sequence
  neoantigenic construct
AMASAAAAEAEKGSPVVVGLLVVGNIIILLSGLSLFAETIWVTADQYRGS

SEQ ID NO: 10 - protein, 76 aa, artificial sequence
  neoantigenic construct
ACFQGLLIFGNVIIGCCGIALTAECIFFVSDQHSLYPLLEATDNDDIYGAAWIGIFVGICLFCLSVLGIVGIMKGS

SEQ ID NO: 11 - protein, 58 aa, artificial sequence
  neoantigenic construct
ATLPLILILLALLSPGAADFNISSLSGLLSPALTESLLVALPPCHLTGGNATLMVRGS

SEQ ID NO: 12 - protein, 62 aa, artificial sequence
  neoantigenic construct
AFGSAVNLQPQLASVTFATNNPTLTTVALEKPLCMFDSKEALTGTHEVYLYVLVDSAISRGS

SEQ ID NO: 13 - protein, 282 aa, artificial sequence
  neoantigenic construct
MGSSHHHHHHSSGSLAEAKVLANRELDKYGVSDYHKNLINNAKTVEGVKDLQAQVVESAKKARISEATDGLSDFLKSQTPAEDTVKSIELAEAKVLANRELDKYGVSDYYKNLINNAKTVEGVKALIDEILAALPGGSAYEGDGGDASRVLEDSNISYGSGGSREPVAATWEASWSEGSKSLDSGGSKATGSPTPRINWLKGGRPLSLGGSTSWLMLPDGINVEVIVVNQVNGGSYILLVGYPPFCDEDQHKLYQQGGSDGNNNLEDDSIVSEDLDVDWSSG

SEQ ID NO: 14 - protein, 282 aa, artificial sequence
  neoantigenic construct
MGSSHHHHHHSSGSLAEAKVLANRELDKYGVSDYHKNLINNAKTVEGVKDLQAQVVESAKKARISEATDGLSDFLKSQTPAEDTVKSIELAEAKVLANRELDKYGVSDYYKNLINNAKTVEGVKALIDEILAALPGGSASQGELIHPKAFPLIVGAQLIHGGSKRKEQEAQEEKRRKQREAQAWGGSMGPGAGRPWPSPNSANSIPYSGGSDRVPNVRVLLTKTLRQTLLEKGGSISLAFFEAASIMRQVSHKHIVGGSSNYQLGELVKLENYPDVIRLISG

SEQ ID NO: 15 - protein, 281 aa, artificial sequence
  neoantigenic construct
MGSSHHHHHHSSGSLAEAKVLANRELDKYGVSDYHKNLINNAKTVEGVKDLQAQVVESAKKARISEATDGLSDFLKSQTPAEDTVKSIELAEAKVLANRELDKYGVSDYYKNLINNAKTVEGVKALIDEILAALPGGSAKVHAVFLDGVKVTLNWHLSSSGGSMMLGPEGGESYVVKLRGLPWGGSKGTIVAQVDSIESFQEFCSTSGGSKYMCNSSCMGVMNRRPILTIIGGSEEKQAAKKRKLEESVEQKRSKGGSTWSLASITYWRPTCANTVSDNSG

SEQ ID NO: 16 - protein, 282 aa, artificial sequence
  neoantigenic construct
MGSSHHHHHHSSGSLAEAKVLANRELDKYGVSDYHKNLINNAKTVEGVKDLQAQVVESAKKARISEATDGLSDFLKSQTPAEDTVKSIELAEAKVLANRELDKYGVSDYYKNLINNAKTVEGVKALIDEILAALPGGSARKRPYSSFSNCKDHREWDHYRGGSRNVMCKKDSPLRTTTIVPPVEGGSHNCLSDPADHRRLTEHVAKAFGGSSAGGWGTEILWSTFAFKASRQGGSPPADFTQPAASAAAAAVAAAAGGSQNAGGSVMIQLVNGSLAVSRASG

SEQ ID NO: 17 - protein, 282 aa, artificial sequence
  neoantigenic construct
MGSSHHHHHHSSGSLAEAKVLANRELDKYGVSDYHKNLINNAKTVEGVKDLQAQVVESAKKARISEATDGLSDFLKSQTPAEDTVKSIELAEAKVLANRELDKYGVSDYYKNLINNAKTVEGVKALIDEILAALPGGSAGEDNRPGMRGCHQMVIDVQTEGGSGQIQEESEGARFKAPPDSTVSGGSSVAAAAAAAVSVVESMVTATEGGSVSHKHIVYLYVVCVRDVENIMGGSEYLKLLHSFVYSVGFVTSPFSGGSDAVASFADVGFVATEEGECSISG

SEQ ID NO: 18 - protein, 282 aa, artificial sequence
  neoantigenic construct
MGSSHHHHHHSSGSLAEAKVLANRELDKYGVSDYHKNLINNAKTVEGVKDLQAQVVESAKKARISEATDGLSDFLKSQTPAEDTVKSIELAEAKVLANRELDKYGVSDYYKNLINNAKTVEGVKALIDEILAALPGGSAVVDHRPKALPVGGFIEEEKDEGGSKQDEYHMVHLLCASRSPPSSPGGSGDTLEEAFEQSAMAMFGYMTDGGSISMSSSKLLLSAKALSTDPASGGSRDLGDEYGWKHVHGDVFRPSSGGSRVSLSHACKNTVKTDAPPEALSG

SEQ ID NO: 19 - protein, 257 aa, artificial sequence
  CMV pp65 construct
MGSSHHHHHHSSGSLAEAKVLANRELDKYGVSDYHKNLINNAKTVEGVKDLQAQVVESAKKARISEATDGLSDFLKSQTPAEDTVKSIELAEAKVLANRELDKYGVSDYYKNLINNAKTVEGVKALIDEILAALPGGSAETRLLQTGIHVRVSQPSLILVGGSIIKPGKISHIMLDVAFTSHEHFGGSWPPWQAGILARNLVPMVATVQGGSRGPQYSEHPTFTSQYRIQGKLGGSQNLKYQEFFWDANDIYRIFAE

(19) United States
(12) Patent Application Publication
(10) Pub. No.: US 2022/0111041 A1
(43) Pub. Date: Apr. 14, 2022

(54) PHAGOCYTISABLE PARTICLE FOR USE IN THE TREATMENT OR PROPHYLAXIS OF CANCER

(71) Applicant: NEOGAP THERAPEUTICS AB, Stockholm (SE)

(72) Inventor: Hans GRONLUND, Lidingö (SE)

(21) Appl. No.: 17/417,601

(22) PCT Filed: Dec. 24, 2019
(86) PCT No.: PCT/EP2019/087029

§ 371 Date: Jun. 23, 2021

(30) Foreign Application Priority Data
Dec. 24, 2018 (GB) 1821205.0

(51) Int. Cl. A61K 39/385; A61K 39/00; A61K 47/69; A61P 35/00

(52) U.S. Cl. CPC A61K 39/385; A61K 39/0011; A61K 47/6933; A61K 2039/645; A61K 2039/55533; A61K 2039/6093; A61K 2039/62; A61P 35/00

(57) ABSTRACT

The invention provides a phagocytosable particle for use in the treatment or prophylaxis of cancer in a subject, wherein the phagocytosable particle comprises a core and a neoantigenic construct tightly associated to the core, and wherein the neoantigenic construct comprises a neoepitope peptide having an amino acid sequence corresponding to an amino acid sequence of a part of a protein or peptide known or suspected to be expressed by a cancer cell in the subject, wherein the part of the protein or peptide has at least one somatic mutated amino acid. The invention also relates to injectable pharmaceutical compositions for use in the treatment or prophylaxis of cancer.

### FIELD OF THE INVENTION

The present invention relates to phagocytosable particles comprising a neoantigenic construct tightly associated to a core for use in the treatment or prophylaxis of cancer. The invention also relates to injectable pharmaceutical compositions for use in the treatment or prophylaxis of cancer.

### BACKGROUND

There are various approaches for modulating the immune system of a subject to treat cancer; such approaches are often referred to as “immunotherapies”. Examples of immunotherapies include immune checkpoint inhibitors, adoptive cell transfer (ACT) therapies, and cancer vaccines.

There has been much success in using immune checkpoint inhibitors for treating cancer, such as the use of monoclonal antibodies that target binding interactions that are important to checkpoints of immune activation. Immune checkpoint inhibitors have been used for the treatment of various cancers, for example in the treatment of melanoma, lung cancer, bladder cancer and gastrointestinal cancers.

There has also been some success with adoptive cell transfer (ACT). For example, a study in which patients with advanced colon cancer were treated using an adoptive immunotherapy protocol was reported by Karlsson et al., Ann Surg Oncol., 2010, 17(7):1747-57. The treatment was based on the isolation and in vitro expansion of autologous tumour-reactive lymphocytes isolated intraoperatively from the first lymph node that naturally drains the tumour (the sentinel node). Sentinel node acquired lymphocytes were collected, activated, expanded against an autologous tumour extract and returned to the patient as a transfusion. No toxic side effects or other adverse effects were observed. Total or marked regression of the disease occurred in four patients with liver and lung metastases and twelve patients displayed partial regression or stable disease.

A significant limitation for ACT is the need to prepare a sufficient quantities of anticancer T-cells, such as tumour-infiltrating lymphocytes (TILs), for administration to a subject. For example, current methods often require the use of invasive surgical procedures to remove a cancer or cancer cells of a subject in order to obtain anticancer T-cells. Furthermore, the cells obtained are few and are frequently unresponsive (anergic) due to immunosuppressive mechanisms from the cancer. This can lead to in vitro expansion being slow, which in turn means that it can take a long time to obtain sufficient quantities of anticancer T-cells for therapeutic use.

Genetically engineered T-cells have been developed to overcome some of the limitations of ACT. Genetically engineered T-cells may be obtained by genetically redirecting a T-cell specificity towards a patient's cancer by introduction of antigen receptors or by introducing a synthetic recognition structure termed a “chimeric antigen receptor” into a T-cell. Although genetically engineered T-cells have found success in treating hematologic cancers, the safety and selectivity of genetically engineered T-cells for treating solid cancers still requires improvement.

An alternative approach to immune checkpoint inhibitors and ACT is the administration of cancer antigens to a subject to elicit an anticancer immune response. Compositions that elicit an anticancer immune response are often referred to as “cancer vaccines”. Cancer vaccines typically comprise a cancer antigen, such as a tumour associated antigen (TAA) or tumour specific antigen (TSA). TAAs are aberrantly expressed by a cancer cell, for example, a TAA may be a protein or peptide that is expressed by both normal cells and cancer cells, but is expressed by cancer cells at a significantly higher level. TSAs are antigens that are expressed by cancer cells and not by normal cells. A particular example of a tumour specific antigen is a neoantigen. A neoantigen is a mutated protein or peptide expressed by a cancer cell, but not a normal cell, that can be bound by a molecule of the immune system such as an antibody or a T-cell receptor (TCR) of a T-cell. The region of the neoantigen that comprises one or more cancer-specific amino acid mutations, and which is known or suspected of being directly bound by a molecule of the immune system, is often referred to as a neoepitope. Therapeutic agents or vaccines that target TSAs, such as neoantigens, are expected to provide more effective and safer cancer treatments.

Despite the interest in immunotherapies for treating cancer, they have to date had limited success. This is due to ineffective modulation or induction of an anticancer immune response, together with challenges associated with the safety and selectivity of the immunotherapy.

Thus, there remains a need for improved immunotherapies for use in the treatment of cancers, which elicit a robust and targeted anticancer immune response, whilst also being suitable for use in a clinical setting.

### SUMMARY OF THE INVENTION

The present invention provides a phagocytosable particle for use in the treatment or prophylaxis of cancer in a subject, wherein the phagocytosable particle comprises a core and a neoantigenic construct tightly associated to the core, and wherein the neoantigenic construct comprises a neoepitope peptide having an amino acid sequence corresponding to an amino acid sequence of a part of a protein or peptide known or suspected to be expressed by a cancer cell in the subject, wherein the part of the protein or peptide has at least one somatic mutated amino acid.

The present invention also provides a method of treating or preventing cancer comprising the step of administering to the subject a phagocytosable particle, wherein the phagocytosable particle comprises a core and a neoantigenic construct tightly associated to the core, and wherein the neoantigenic construct comprises a neoepitope peptide having an amino acid sequence corresponding to an amino acid sequence of a part of a protein or peptide known or suspected to be expressed by a cancer cell in the subject, wherein the part of the protein or peptide has at least one somatic mutated amino acid.

The present invention also provides the use of a phagocytosable particle for the manufacture of a medicament for the treatment or prophylaxis of cancer, wherein the phagocytosable particle comprises a core and a neoantigenic construct tightly associated to the core, and wherein the neoantigenic construct comprises a neoepitope peptide having an amino acid sequence corresponding to an amino acid sequence of a part of a protein or peptide known or suspected to be expressed by a cancer cell in the subject, wherein the part of the protein or peptide has at least one somatic mutated amino acid.

The present invention also provides an injectable pharmaceutical composition comprising a phagocytosable particle wherein the phagocytosable particle comprises a core and a neoantigenic construct tightly associated to the core, and wherein the neoantigenic construct comprises a neoepitope peptide having an amino acid sequence corresponding to an amino acid sequence of a part of a protein or peptide known or suspected to be expressed by a cancer cell in the subject, wherein the part of the protein or peptide has at least one somatic mutated amino acid.

The present invention further provides a phagocytosable particle of the invention for use in the treatment or prophylaxis of cancer in a subject, or an injectable pharmaceutical composition of the invention for use in the treatment or prophylaxis of cancer in a subject, or a method for the treatment or prophylaxis of cancer in a subject of the invention, wherein the treatment or prophylaxis of cancer further comprises the step of:

administering one or more subsequent doses of the phagocytosable particle or injectable pharmaceutical composition to the subject, wherein the subject is one whom has previously been administered a dose of the phagocytosable particle or injectable pharmaceutical composition sufficient to elicit an immune response towards a cancer cell in the subject.

The present invention further provides a phagocytosable particle of the invention for use in the treatment or prophylaxis of cancer in a subject, or an injectable pharmaceutical composition of the invention for use in the treatment or prophylaxis of cancer in a subject, or a method for the treatment or prophylaxis of cancer in a subject of the invention, wherein the treatment or prophylaxis of cancer further comprises the step of:
(a) harvesting APCs and anticancer T-cells from the subject after the administration of the phagocytosable particle to the subject;
(b) expanding the anticancer T-cells harvested from the subject; and
(c) administering a therapeutic dose of the expanded anticancer T-cells to the subject.

The present inventors have found that following administration to a subject, the phagocytosable particles described herein are internalised by antigen-presenting cells (APCs). The associated neoantigenic constructs are then presented on the surface of the APCs, and bring about activation and expansion of anticancer T-cells in the subject. Use of the phagocytosable particles leads to a surprisingly high uptake of neoantigenic constructs and subsequent presentation of a wide variety of neoepitopes on the surface of the APCs. The inventors have shown in a mouse model that administration of phagocytosable particles comprising two types of neoantigenic construct by injection into an inguinal lymph node or subcutaneously resulted in a dose dependent increase in anti-neoepitope antibodies in blood serum samples taken from the mice. The same mice were subsequently injected with melanoma cancer cells (B16F10). Advantageously, the inventors found that administration of the phagocytosable particles to the mice before injection of the cancer cells results in a dose dependent prophylactic effect on tumour growth in the mice. Thus, the present inventors have found that by administering phagocytosable particles as described herein to a subject, a robust anticancer immune response can be elicited in the subject, and the phagocytosable particles of the invention may be successfully used as a prophylactic vaccination for cancer to reduce cancer growth.

In addition, the inventors have also shown in a mouse xenograft model of colorectal cancer, that administration of a phagocytosable particle composition comprising six types of neoantigenic construct, before and after transplantation of colon cancer cells (MC-38 cell line), resulted in a robust anticancer immune response that inhibited tumour growth in the mice. Thus, the present inventors have shown in two mouse models of cancer the therapeutic and prophylactic potential of the phagocytosable particles of the invention.

The present inventors have also found that the core of a phagocytosable particle (e.g. a polymer particle) as described herein acts as a very effective carrier for the neoantigenic constructs. Without wishing to be bound by any particular theory, it is believed that phagocytosable particles as defined herein are especially effective because the entire phagocytosable particle, including the core and tightly associated neoantigen construct, are internalised by APCs by phagocytosis into a phagosome. The neoantigenic constructs are then cleaved from the core of the particle and processed in the phagosome. Fragments of the neoantigenic construct are then presented on the surface of the APC via the major histocompatibility (MHC) class II pathway and presented on the cell surface by a MHC class II molecule. It is also believed that this is not the exclusive process for the neoepitope to be presented on the surface of an APC, and that some fragments of the neoantigenic construct may also be presented on the surface of APCs via the major histocompatibility (MHC) class I pathway and presented on the cell surface by a MHC class I molecule, in a process known as cross-presentation. Thus, although it is expected that fragments of the neoantigenic construct are presented on APCs predominantly via the MHC class II pathway, it is expected that some will be presented via the MHC class I pathway, and so the present invention harnesses both pathways to a varying extent.

When antigens are presented by an MHC class II molecule, they generally activate helper T-cells (also known as CD4+ T-cells), which predominantly orchestrate immune responses by secretion of cytokines, inducing class switching of B-cells to assist the B-cells to make antibodies and stimulating activation and expansion of other T-cell types, in particular cytotoxic T-cells (e.g. CD8+ T-cells) and memory T-cells (e.g. CD8+ memory T-cells). In addition, CD4+ T-cells can directly kill other cell types (Borst et al. Nat Rev Immunol, 2018, 18(10), 635-647). This means that by administering the phagocytosable particles as defined herein to a subject, it is possible to activate multiple types of immune cells, which results in an anticancer immune response that starts slowly (thus leading to few side effects), has a long lasting effect, and can target the cancers in many different ways by harnessing the whole immune system (rather than only activating CD8+ T-cell which can only attack the tumour cells directly). This is in contrast to what would be expected to occur when an antigen (e.g. a neoantigen) is provided as free peptide or a nucleotide construct that expresses the peptide. Such an antigen would be expected to be taken up into the cytosol of an APC, which results in the neoantigen being presented on the cell surface solely via the MHC class I pathway by an MHC class I molecule. This, in turn, results predominantly in the activation of CD8+ T-cells. Furthermore, after inducing an anticancer immune response, memory T-cells derived from the anticancer T-helper cells remain in circulation and they can mount a rapid and effective secondary immune response for as long as cancer cells expressing the neoepitope remain in the body, or if the same cancer returns.

The present inventors have also found that phagocytosable particles as described herein can be efficiently purified and sterilised, thus removing contaminants, such as pathogens (e.g. bacteria, fungus and viruses), endotoxins and other antigenic contaminants from the phagocytosable particles before administration to a subject. This is particularly advantageous because the removal of contaminants from the phagocytosable particles as described herein reduces non-specific immune responses in the subject after administration, and therefore improves safety and efficacy of the phagocytosable particles.

The inventors also understand the phagocytosable particles to be well tolerated in a subject after administration, in part, due to the inert properties of the core and high sterility of the phagocytosable particles.

### BRIEF DESCRIPTION OF THE FIGURES

FIG. 1 shows the effect of phagocytosable particle size on T-cell activation. A proliferation assay (with thymidine incorporation) was used to assess the number of splenocytes obtained from ovalbumin sensitized mice. Comparison of ovalbumin coupled to differently sized phagocytosable particle with a diameter of 5.6 μm, 1 μm and 0.2 μm are shown. P-values determined using students T-test and written indicated when p<0.05 found. Staples denote SD.

FIG. 2 shows the expansion of T-cells by stimulation with neoepitopes, NA1-9. FIG. 2A shows the number of cells in the culture over time. FIG. 2B shows the % CD4+/total T-cells.

FIG. 2C shows the T-bet expression in CD4+ T-cells. FIG. 2D shows the expression of Granzyme B and Perforin in CD8+ T-cells.

FIG. 3 shows the expansion of T-cells by stimulation with a neoantigenic construct. Percent among T-cells (small squares) and total number (large squares) of CD4+ T-cells, as well as proliferating CD4+ cells (circles) are shown.

FIG. 4 shows the expansion of anticancer T-cells by stimulation with a neoantigenic construct. FIG. 4A shows the number of cells in PBMC culture over time (days). PBMC culture contains various cell types, including APCs and T-cells. The top line (Pat2 personalised NA) in FIG. 4A shows the number of cells in the PBMC culture after incubation with with phagocytosable particles comprising a polystyrene core (MyOne™ Carboxylic Acid Dynabeads®) and a personalised neoantigenic construct tightly associated to the core (SEQ ID NO: 3). The two lines at the bottom of the FIG. 4A (Pat2 NA 1+3 and Pat 2 NA 4+5) display the number of cells in two separate PBMC cultures after incubation with phagocytosable particles comprising a polystyrene core (MyOne™ Carboxylic Acid Dynabeads®) and predicted neoantigenic constructs tightly associated to the core. FIG. 4B shows the % CD4+/total T-cells. FIG. 4C visualizes an analysis performed with the Barnes-Hut Stochastic Neighbor Embedding (BH-SNE) algorithm for CD4+ T-cells, where all cells in the samples are clustered on a 2-dimensional map according to the similarity in expression intensity according to a set of chosen markers: CD28, CD57, T-bet, GATA-3, Perforin, Granzyme B (GZB), Ki-67 and PD-1.

FIG. 5A shows the confocal microscope images of PBMCs with intracellular, phagocytosed particles. Three sizes of phagocytosable particle are shown (4.5 μm, 2.8 μm or 1 μm) following incubation for 18 h at 37° C. of PBMCs with the phagocytosed particles.

FIG. 5B shows the cellular uptake of phagocytosable particles of two sizes (4.5 μm or 2.8 μm) after incubation with PBMCs for 18 h at 37° C., as assessed by manual counting.

FIG. 5C shows the cellular uptake of phagocytosable particles of three sizes (4.5 μm, 2.8 μm or 1 μm) after incubation in PBMCs for 18 h at 37° C., as assessed by volume calculation (*p<0.05 **p<0.01 ***p<0.001, calculated using Student's T-test).

FIG. 6A shows the relative increase in the level of IFNγ-production in PBMCs from CMV-sensitive healthy donors (n=2) stimulated with phagocytosable particles of three sizes (4.5 μm, 2.8 μm or 1 μm) compared to non-stimulated cells, as assessed in the FluoroSpot assay of Example 3a(iv).

FIG. 6B shows the relative increase in the level of IL22-production in PBMCs from a CMV-sensitive healthy donor (n=1) stimulated with phagocytosable particles of three sizes (4.5 μm, 2.8 μm or 1 μm) compared to non-stimulated cells, as assessed in the FluoroSpot assay of Example 3a(iv).

FIG. 6C shows the relative increase in the level of IL17-production in PBMCs from CMV-sensitive healthy donor (n=1) stimulated with phagocytosable particles of three sizes (4.5 μm, 2.8 μm or 1 μm) compared to non-stimulated cells, as assessed in the FluoroSpot assay of Example 3a(iv).

FIG. 6D shows the relative increase in the dual-cytokine production of IFNγ and IL-17 in PBMCs from a CMV-sensitive healthy donor (n=1) stimulated with phagocytosable particles of three sizes (4.5 μm, 2.8 μm or 1 μm) compared to non-stimulated cells, as assessed in the FluoroSpot assay of Example 3a(iv).

FIG. 6E shows the relative increase in the dual-cytokine production of IL22 and IL17 in PBMCs from a CMV-sensitive healthy donor (n=1) stimulated with phagocytosable particles of three sizes (4.5 μm, 2.8 μm or 1 μm) compared to non-stimulated cells, as assessed in the FluoroSpot assay of Example 3a(iv).

FIGS. 7A, 7B, 7C and 7D show the proportion of anti-neoepitope antibodies in blood serum samples taken from mice following low or high doses of two species of phagocytosable particles administered into an inguinal lymph node or subcutaneously (n=3 for each dose and route of administration). The two species of phagocytosable particles were 1) polystyrene particles tightly associated to neoantigenic construct M272120 (SEQ ID NO: 1); and 2) polystyrene particles tightly associated to neoantigenic construct M304748 (SEQ ID NO: 2). FIGS. 7A and 7B show the proportion of anti-neoepitope antibodies to neoantigenic construct M272120 (SEQ ID NO: 1) (FIG. 7A) or neoantigenic construct M304748 (SEQ ID NO: 2) (FIG. 7B) in blood serum harvested from mice 22 days following a first dose of the phagocytosable particles; and FIGS. 7C and 7D show the proportion of anti-neoepitope antibodies to neoantigenic construct M272120 (SEQ ID NO: 1) (FIG. 7C) or neoantigenic construct M304748 (SEQ ID NO: 2) (FIG. 7D) in blood serum harvested from mice 23 days following a second dose of the phagocytosable particles around a month after the first dose of phagocytosable particles. As a control, blood serum samples taken from naïve mice (n=3, no dose of phagocytosable particles administered) were also analysed. Mice that received one or two high doses of phagocytosable particles (administered into an inguinal lymph node or subcutaneously) had a greater proportion of anti-neoepitope antibodies in blood serum samples compared to the mice that received one or two low doses of phagocytosable particles via the same route, and the mice that did not receive a dose of phagocytosable particles (i.e. the naïve mice).

FIG. 8 shows the change in tumour volume over time (days, D) after injection of melanoma cancer cell line B16F10 into mice that were previously administered two doses of phagocytosable particles (polystyrene particles tightly associated to M272120, and polystyrene particles tightly associated to M304748). The change in tumour volume over time is shown for mice (n=3) previously administered the following doses of phagocytosable particles: two low doses of phagocytosable particles into an inguinal lymph node (squares, ▪); two high doses of phagocytosable particles injected into an inguinal lymph node (triangles, ▾); two low doses of phagocytosable particles injected subcutaneously (triangles, ▴); two high doses of phagocytosable particles injected subcutaneously (diamonds, ♦). Administration of phagocytosable particles shows a dose dependent prophylactic effect on tumour volume.

FIG. 9 shows the tumour volume in mice (n=5) following administration with a first and second dose of a phagocytosable particle composition comprising six different groups of phagocytosable particle, wherein each group comprised a core coupled to a different MC38 neoantigen construct (SEQ ID NOs. 13-18). The first dose was administered on Day −5, (time point A) and the second dose was administered on Day 13 (time point C). Mice were implanted with the MC38 tumour cells on Day 0 (time point B). Tumour progression was compared with a non-vaccinated group (negative control, n=5). The differences in tumour volume at the end of the experiment was calculated with student's t-test. ***p<0.001.

### DETAILED DESCRIPTION OF THE INVENTION

The Neoantigenic Construct and Neoepitope Peptide

The phagocytosable particle for use in the present invention comprises a neoantigenic construct tightly associated to a core. The neoantigenic construct of the invention comprises a neoepitope peptide.

A neoepitope peptide for use in the present invention is a peptide having an amino acid sequence corresponding to an amino acid sequence of a part of a protein or peptide known or suspected to be expressed by a cancer cell in the subject, wherein the part of the protein or peptide has at least one somatic mutated amino acid. A “somatic mutated amino acid” of a neoepitope peptide is an amino acid that is different or not present in the part of the protein or peptide corresponding to the neoepitope peptide amino acid sequence when that part of the protein or peptide is expressed by a non-cancerous cell (e.g. a somatic cell). For example, a “somatic mutated amino acid” of a neoepitope peptide may be a deletion (i.e. an amino acid that has been deleted), an addition (i.e. an amino acid that has been added) or a substitution (i.e. an amino acid that has been substituted for a different amino acid). Such somatic mutated amino acids may also be referred to as a “cancer-specific somatic mutated amino acid” because the somatic mutated amino acids are present in a cancer cell, but not in a normal cell (e.g. a somatic cell). Preferably, the somatic mutated amino acid(s) of a neoepitope peptide is/are a substitution (i.e. one or more amino acid has been substituted for a different amino acid).

Mutated somatic amino acids in a protein or peptide expressed by a cell can occur as a result of infidelity of DNA replication occurring at each cell division creating substitutions, deletions or insertions of nucleotides into the DNA of a cell. Nucleotide substitutions can result in a different amino acid being coded for compared to the amino acid coded for by the somatic non-mutated nucleic acid sequence, thus resulting in a different amino acid in the protein/peptide compared to the protein/peptide in a normal non-cancerous cell (e.g. a somatic cell). Nucleotide insertion(s) and/or deletion(s) can result in a reading frame error (i.e. a “frameshift mutation”), thus resulting in a new amino acid sequence at the protein level (i.e. nucleotide insertion(s) or deletion(s) altering the reading frame of the DNA and thus altering most or all of the amino acids encoded by the DNA after the mutation compared to a normal cell (e.g. somatic cell)). Additionally, or alternatively, an insertion and/or deletion can result in the introduction of a stop codon, thus resulting in a truncated protein at the protein level. A nucleotide substitution can individually alter codon(s) and result in amino acid substitution(s) at the protein level and/or the introduction of a stop codon, thus resulting in a truncated protein at the protein level.

Neoepitope peptides for use in the present invention are peptides having an amino acid sequence corresponding to an amino acid sequence of a part of a protein or peptide known or suspected to be expressed by a cancer cell in the subject, wherein the part of the protein or peptide has at least one somatic mutated amino acid (e.g. 1, 2, 3, 4, or 5, or more, somatic mutated amino acids).

A mutated protein or peptide known or suspected to be expressed by a cancer cell in a subject may also be referred to as a “cancer-specific mutated protein or peptide”. That is because the mutated protein or peptide is known or suspected to be expressed in a cancer cell, but not a normal cell (e.g. a somatic cell).

A cancer-specific mutated protein or peptide, and its amino acid sequence that a neoepitope peptide amino acid sequence can correspond to, may be identified using a variety of techniques. For example, a cancer-specific mutated protein or peptide, and its amino acid sequence including its cancer-specific somatic mutated amino acid(s), may be identified from publicly available protein databases, such as the COSMIC database (Forbes et al., Nucleic Acids Res, 45(D1), D777-D783, the database is accessible at http://cancer.sanger.ac.uk/cosmic). Somatic mutated amino acid sequences identified from the COSMIC database, or similar databases, are referred to herein as “predicted neoepitope peptides”. Neoantigenic constructs consisting of one or more “predicted neoepitope peptides” are referred to herein as a “predicted neoantigenic constructs”.

In an alternative or additional approach to identify a cancer-specific mutated protein or peptide, and its amino acid sequence that a neoepitope peptide amino acid sequence can correspond to the genome, exome transcriptome and/or proteome of a cancer cell obtained from a cancer in a subject may be established, and thus the mutations in a cancer cell deduced. That can be done, for example, by comparison of the proteome, genome, exome or transcriptome derived data with reference nucleotide sequences or amino acid sequence. Suitable reference sequences may be obtained from the genome, exome or transcriptome of a non-cancerous cell (e.g. a somatic cell) obtained from the subject or from publicly available nucleotide or protein databases, such as the UniProt databases (https://www.uniprot.org/) and the EBI expression atlas (https://www.ebi.ac.uk/gxa/home), which provides information on proteins and peptides which are expressed in tissues and cancer cell lines. In an alternative or additional approach, a cancer-specific mutated protein or peptide, and its amino acid sequence that a neoepitope peptide amino acid sequence can correspond to, may be one that has been previously identified by analysis of the genome, exome, transcriptome and/or proteome of a tumour of a subject. Suitable techniques for sequencing the genome, exome or transcriptome of a cancer cell or a normal cell are known in the art, and include, for example Sanger sequencing and next-generation sequencing. Suitable techniques for obtaining proteome data include Multiple Reaction Monitoring (MRM) mass spectrometry. Somatic mutated amino acid sequences identified from genome, exome, transcriptome or proteome data obtained from a subject are referred to herein as “personalised neoepitope peptides”. Neoantigenic constructs consisting of one or more “personalised neoepitope peptides” are referred to herein as a “personalised neoantigenic constructs”.

A neoepitope peptide for use in the present invention has one or more somatic mutated amino acids. For example, it may have one somatic mutated amino acid, or more than one somatic mutated amino acids, i.e. from two to all of the amino acids in the part of the protein or peptide may be mutated. For example, a neoepitope peptide of the invention may have 1 to 10 (for example 1, 2, 3, 4, 5, 6, 7, 8, 9 or 10) somatic mutated amino acids, more preferably 1 to 8 (for example 1, 2, 3, 4, 5, 6, 7 or 8) mutated amino acids; or, for example, 1 to 6 (for example 1, 2, 3, 4, 5 or 6) mutated amino acids; or, for example, 1 to 5 (for example 1, 2, 3, 4 or 5) somatic mutated amino acids; or, for example, 1 to 4 (for example 1, 2, 3, or 4) somatic mutated amino acids. In a preferred embodiment of the invention, a neoepitope peptide of the invention has 1, 2 or 3 somatic mutated amino acids. In one preferred embodiment, a neoepitope peptide of the invention has 1 or 2 somatic mutated amino acids. Even more preferably, a neoepitope peptide of the invention has one somatic mutated amino acid.

In one preferred embodiment, a neoepitope peptide of the invention has most or all somatic mutated amino acids. Even more preferably, a neoepitope peptide of the invention has all somatic mutated amino acids. Such neoepitope peptides having most or all somatic mutated amino acids may correspond to a part of a protein or a peptide resulting from a frameshift type mutation in the DNA of a cell.

The one or more amino acid mutations of a neoepitope peptide for use in the present invention may be located at any amino acid position within the neoepitope peptide amino acid sequence. In one preferred embodiment, at least one of the somatic mutated amino acids (or the one somatic mutated amino acid in embodiments where there is only one somatic mutated amino acid in the neoepitope peptide) is located in the central portion of the neoepitope peptide. For example, when a neoepitope peptide amino acid sequence is at least 3 amino acids in length (for example at least 5 amino acids in length or at least 7 amino acids in length), the central portion of the neoepitope peptide is the central 1 amino acid of the sequence when the neoepitope peptide has an odd number of amino acids in its sequence; or the central 2 amino acids when the neoepitope peptide has an even number of amino acids in its sequence. For example, when a neoepitope peptide amino acid sequence is at least 9 amino acids in length, the central portion of the neoepitope peptide is the central 3 amino acids of the sequence (and preferably the 1 central amino acid) when the neoepitope peptide has an odd number of amino acids in its sequence; or the central 4 amino acids (and preferably the 2 central amino acid) when the neoepitope peptide has an even number of amino acids in its sequence. For example, when a neoepitope peptide amino acid sequence is at least 11 amino acids in length, the central portion of the neoepitope peptide is the central 5 amino acids of the sequence (and preferably the 1 central amino acid) when the neoepitope peptide has an odd number of amino acids in its sequence; or the central 6 amino acids when the neoepitope peptide has an even number of amino acids in its sequence. More preferably, when a neoepitope peptide amino acid sequence is at least 11 amino acids in length, the central portion of the neoepitope peptide is the central 3 amino acids (and preferably the 1 central amino acid) of the sequence when the neoepitope peptide has an odd number of amino acids in its sequence; or the central 4 amino acids (and preferably the 2 central amino acid) when the neoepitope peptide has an even number of amino acids in its sequence.

In one preferred embodiment, at least one of the somatic mutated amino acids (or the one somatic mutated amino acid in embodiments where there is only one somatic mutated amino acid in the neoepitope peptide) of a neoepitope peptide is located at the central position of the neoepitope peptide when the neoepitope peptide has an odd number of amino acids in its sequence, or at either of the two most central positions of the amino acid sequence when the neoepitope peptide has an even number of amino acids in its sequence.

In certain embodiments of the invention, most or all of the amino acids of the neoepitope peptide are somatic mutated amino acids. In such embodiments the somatic mutated amino acids in the protein or peptide expressed by a cancer cell may have occurred due to an error in the reading frame of the encoding DNA (i.e. due to a frameshift mutation) resulting in all or most of the amino acids in the part of the protein or peptide being different to the part of the protein or peptide expressed in a normal non-cancerous cell.

A neoepitope peptide of the invention may have an amino acid sequence that is 3 to 200 amino acids in length (for example 3, 4, 5, 6, 7, 8, 9, 10, 11, 12, 13, 14, 15, 16, 17, 18, 19, 20, 21, 22, 23, 24, 25, 26, 27, 28, 29, 30, 50, 75, 100, 125, 150, 175 or 200 amino acids in length). Preferably a neoepitope peptide of the invention may have an amino acid sequence that is 3 to 50 amino acids in length (for example 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45 or 50 amino acids in length), more preferably 3 to 30 amino acids in length (for example 3, 4, 5, 6, 7, 8, 9, 10, 11, 12, 13, 14, 15, 16, 17, 18, 19, 20, 21, 22, 23, 24, 25, 26, 27, 28, 29 or 30 amino acids in length), more preferably 3 to 25 amino acids (for example 3, 4, 5, 6, 7, 8, 9, 10, 11, 12, 13, 14, 15, 16, 17, 18, 19, 20, 21, 22, 23, 24, or 25 amino acids in length), more preferably 5 to 25 amino acids (for example 5, 6, 7, 8, 9, 10, 11, 12, 13, 14, 15, 16, 17, 18, 19, 20, 21, 22, 23, 24, or 25 amino acids in length), more preferably 8 to 25 amino acids (for example 8, 9, 10, 11, 12, 13, 14, 15, 16, 17, 18, 19, 20, 21, 22, 23, 24, or 25 amino acids in length), and even more preferably 11 to 25 amino acids in length (for example 10, 11, 12, 13, 14, 15, 16, 17, 18, 19, 20, 21, 22, 23, 24, or 25 amino acids in length). In one preferred embodiment, a neoepitope peptide of the invention has 3 to 25 amino acids, 5 to 25 amino acids, 10 to 25 amino acids, 11 to 25 amino acids, 12 to 25 amino acids, 13 to 25 amino acids, 15 to 25 amino acids, 17 to 25 amino acids, 19 to 25 amino acids, 20 to 25 amino acids in length, or 21 to 25 amino acids in length. In another preferred embodiment, a neoepitope peptide of the invention has 3 to 23 amino acids, 5 to 23 amino acids, 10 to 23 amino acids, 11 to 23 amino acids, 12 to 23 amino acids, 13 to 23 amino acids, 15 to 23 amino acids, 17 to 23 amino acids, 19 to 23 amino acids, 20 to 23 amino acids, or 21 to 23 amino acids in length. In another preferred embodiment, a neoepitope peptide of the invention is 3 to 21 amino acids, 5 to 21 amino acids, 10 to 21 amino acids, 11 to 21 amino acids, 12 to 21 amino acids, 13 to 21 amino acids, 15 to 21 amino acids, 17 to 21 amino acids, or 19 to 21 amino acids in length. In another preferred embodiment, a neoepitope peptide of the invention is 3 to 19 amino acids, 5 to 19 amino acids, 10 to 19 amino acids, 11 to 19 amino acids, 12 to 21 amino acids, 13 to 21 amino acids, 15 to 21 amino acids, or 17 to 19 amino acids in length. In another preferred embodiment, a neoepitope peptide of the invention has 3 to 17 amino acids, 5 to 17 amino acids 10 to 17 amino acids, 11 to 17 amino acids, 12 to 17 amino acids, 13 to 17 amino acids or 15 to 17 amino acids in length. In another preferred embodiment, a neoepitope peptide of the invention has 3 to 15 amino acids, 3 to 15 amino acids 10 to 15 amino acids, 11 to 15 amino acids, 12 to 15 amino acids or 13 to 15 amino acids in length. In another preferred embodiment, a neoepitope peptide of the invention is 3 to 19 amino acids, 5 to 17 amino acids, 5 to 15 amino acids, 5 to 13 amino acids, or 5 to 10 amino acids in length. In another preferred embodiment, a neoepitope peptide of the invention is 3 to 19 amino acids, 5 to 17 amino acids, 3 to 15 amino acids, 3 to 10 amino acids, or 5 to 10 amino acids in length. In another preferred embodiment, a neoepitope peptide of the invention is 3 to 19 amino acids, 3 to 17 amino acids, 3 to 13 amino acids, 3 to 10 amino acids, or 3 to 7 amino acids in length.

In preferred embodiments of the invention, the neoepitope peptide is 10 to 25 amino acids, 10 to 23 amino acids, 10 to 21 amino acids, 10 to 19 amino acids, 10 to 17 amino acids, 10 to 15 amino acids and comprises 1, 2, 3, 4, or 5, or all, somatic mutated amino acids. Preferably, the neoepitope peptide is 10 to 25 amino acids, 10 to 23 amino acids, 10 to 21 amino acids, 10 to 19 amino acids, 10 to 17 amino acids, 10 to 15 amino acids in length and comprises 1, 2 or 3 somatic, or all, mutated amino acids. More preferably, the neoepitope peptide is 10 to 25 amino acids, 10 to 23 amino acids, 10 to 21 amino acids, 10 to 19 amino acids, 10 to 17 amino acids, 10 to 15 amino acids in length and the neoepitope comprises 1 or 2 somatic, or all, mutated amino acids. Even more preferably, the neoepitope peptide is 10 to 25 amino acids, 10 to 23 amino acids, 10 to 21 amino acids, 10 to 19 amino acids, 10 to 17 amino acids, 10 to 15 amino acids in length and comprises one somatic, or all, mutated amino acid. Even more preferably, the neoepitope peptide is 10 to 25 amino acids, 10 to 23 amino acids, 10 to 21 amino acids, 10 to 19 amino acids, 10 to 17 amino acids, 10 to 15 amino acids in length and comprises one somatic mutated amino acid.

In another preferred embodiments of the invention, the neoepitope peptide is 3 to 25 amino acids, 3 to 17 amino acids, 3 to 15 amino acids, 3 to 10 amino acids, or 5 to 10 amino acids in length, and comprises 1, 2, 3, 4, or 5, or all, somatic mutated amino acids. Preferably, the neoepitope peptide is 3 to 25 amino acids, 3 to 17 amino acids, 3 to 15 amino acids, 3 to 10 amino acids, or 5 to 10 amino acids in length, and comprises 1, 2 or 3, or all, somatic mutated amino acids. More preferably, the neoepitope peptide is 3 to 25 amino acids, 3 to 17 amino acids, 3 to 15 amino acids, 3 to 10 amino acids, or 5 to 10 amino acids in length, and comprises 1 or 2, or all, somatic mutated amino acids. Even more preferably, the neoepitope peptide is 3 to 25 amino acids, 3 to 17 amino acids, 3 to 15 amino acids, 3 to 10 amino acids, or 5 to 10 amino acids in length, and comprises one, or all, somatic mutated amino acid. Even more preferably, the neoepitope peptide is 3 to 25 amino acids, 3 to 17 amino acids, 3 to 15 amino acids, 3 to 10 amino acids, or 5 to 10 amino acids in length, and comprises one, or all, somatic mutated amino acid.

The present inventors have advantageously found that neoepitopes of the invention, particularly those having an amino acid sequence that corresponds to an amino acid sequence of a part of a cancer-specific protein or peptide that is 3 to 25 amino acids in length and comprising one or more somatic mutated amino acids, are particularly effective at eliciting an anticancer immune response in a subject, whilst not eliciting an autoimmune or non-cancer specific immune response in the subject.

The neoantigenic construct may comprise one neoepitope peptide, or it may comprise more than one neoepitope peptide. For example, the neoantigenic construct may comprise 1 to 50 neoepitope peptides (e.g. 1, 2, 3, 4, 5, 6, 7, 8, 9, 10, 11, 12, 13, 14, 15, 16, 17, 18, 19, 20, 21, 22, 23, 24, 25, 23, 24, 25, 26, 27, 28, 29, 30, 31, 32, 33, 34, 35, 36, 37, 38, 39, 40, 41, 42, 43, 44, 45, 46, 47, 48, 49, 50 neoepitope peptides). Preferably, the neoantigenic construct comprises 1 to 20 neoepitope peptides (e.g. 1, 2, 3, 4, 5, 6, 7, 8, 9, 10, 11, 12, 13, 14, 15, 16, 17, 18, 19 or 20 neoepitope peptides), more preferably, 1 to 15 neoepitope peptides (e.g. 1, 2, 3, 4, 5, 6, 7, 8, 9, 10, 11, 12, 13, 14 or 15, neoepitope peptides), more preferably 1 to 10 neoepitope peptides (e.g. 1, 2, 3, 4, 5, 6, 7, 8, 9 or 10), more preferably 1 to 8 neoepitope peptides (e.g. 1, 2, 3, 4, 5, 6, 7 or 8), more preferably 1 to 6 neoepitope peptides (e.g. 1, 2, 3, 4, 5 or 6), and even more preferably 1 to 5 neoepitope peptides (e.g. 1, 2, 3, 4, or 5). It is especially preferred that the neoantigenic construct comprises 1 to 5 neoepitope peptides (e.g. 1, 2, 3, or 4), and even more preferably 3 to 5 neoepitope peptides, for example 3, 4 or 5 neoepitope peptides.

In one embodiment of the invention, the neoantigenic construct comprises two or more neoepitope peptides. For example, the neoantigenic construct may comprise 2 to 50 neoepitope peptides (e.g. 2, 3, 4, 5, 6, 7, 8, 9, 10, 11, 12, 13, 14, 15, 16, 17, 18, 19, 20, 21, 22, 23, 24, 25, 23, 24, 25, 26, 27, 28, 29, 30, 31, 32, 33, 34, 35, 36, 37, 38, 39, 40, 41, 42, 43, 44, 45, 46, 47, 48, 49, 50 neoepitope peptides). Preferably, the neoantigenic construct may comprise 2 to 20 neoepitope peptides (e.g. 2, 3, 4, 5, 6, 7, 8, 9, 10, 11, 12, 13, 14, 15, 16, 17, 18, 19 or 20 neoepitope peptides), more preferably, the neoantigenic construct may comprise 2 to 15 neoepitope peptides (e.g. 2, 3, 4, 5, 6, 7, 8, 9, 10, 11, 12, 13, 14 or 15, neoepitope peptides), more preferably 2 to 10 neoepitope peptides (e.g. 2, 3, 4, 5, 6, 7, 8, 9 or 10), more preferably 2 to 8 neoepitope peptides (e.g. 2, 3, 4, 5, 6, 7 or 8), more preferably 2 to 6 neoepitope peptides (e.g. 2, 3, 4, 5 or 6), and even more preferably 2 to 5 neoepitope peptides (e.g. 2, 3, 4, or 5). It is especially preferred that the neoantigenic construct comprises 2 to 5 neoepitope peptides (e.g. 2, 3, 4, or 5), and even more preferably 3 to 5 neoepitope peptides, for example 3, 4 or 5 neoepitope peptides.

In another embodiment of the invention, the neoantigenic construct comprises three or more neoepitope peptides. For example, the neoantigenic construct may comprise 3 to 50 neoepitope peptides (e.g. 3, 4, 5, 6, 7, 8, 9, 10, 11, 12, 13, 14, 15, 16, 17, 18, 19, 20, 21, 22, 23, 24, 25, 23, 24, 25, 26, 27, 28, 29, 30, 31, 32, 33, 34, 35, 36, 37, 38, 39, 40, 41, 42, 43, 44, 45, 46, 47, 48, 49, 50 neoepitope peptides).Preferably, the neoantigenic construct may comprise 3 to 20 neoepitope peptides (e.g. 3, 4, 5, 6, 7, 8, 9, 10, 11, 12, 13, 14, 15, 16, 17, 18, 19 or 20 neoepitope peptides), more preferably, the neoantigenic construct may comprise 3 to 15 neoepitope peptides (e.g. 3, 4, 5, 6, 7, 8, 9, 10, 11, 12, 13, 14 or 15, neoepitope peptides), more preferably 3 to 10 neoepitope peptides (e.g. 3, 4, 5, 6, 7, 8, 9 or 10), more preferably 3 to 8 neoepitope peptides (e.g. 3, 4, 5, 6, 7 or 8), more preferably 3 to 6 neoepitope peptides (e.g. 3, 4, 5 or 6), and even more preferably 3 to 5 neoepitope peptides (e.g. 3, 4, or 5). It is especially preferred that the neoantigenic construct comprises 3 to 5 neoepitope peptides (e.g. 3, 4 or 5), and even more preferably 5 neoepitope peptides.

In a further embodiment of the invention, the neoantigenic construct comprises four or more neoepitope peptides (e.g. 4 neoepitope peptides), five or more neoepitope peptides (e.g. 5 neoepitope peptides), or six or more neoepitope peptides (e.g. 6 neoepitope peptides).

For the avoidance of doubt, in embodiments where the neoantigenic construct may comprise more than one neoepitope peptide (for example, one or more neoepitope peptides, two or more neoepitope peptides, or three or more neoepitope peptides), each neoepitope peptide is a neoepitope peptide as described herein for use in the present invention (i.e. each neoepitope peptide of a neoantigenic construct has an amino acid sequence corresponding to an amino acid sequence of a part of a protein or peptide known or suspected to be expressed by a cancer cell in the subject, wherein the part of the protein or peptide has at least one somatic mutated amino acid). In such embodiments, each neoepitope peptide may independently have any of the properties and/or characteristics of a neoepitope peptide described herein.

In embodiments where the neoantigenic construct may comprise more than one neoepitope peptide (e.g. one or more neoepitope peptides, two or more neoepitope peptides or three or more neoepitope peptides), each neoepitope peptide may have the same amino acid sequence, or the neoepitope peptides may have different amino acid sequences (i.e. some neoepitope peptides of a neoantigenic construct may have different amino acid sequences, or all of the neoepitope peptides of a neoantigenic construct may have different amino acid sequences).

In certain preferred embodiments where the neoantigenic construct may comprise more than one neoepitope peptide, some or all of the neoepitope peptides have different amino acid sequences, and more preferably all of the neoepitope peptides have different amino acid sequences. In another embodiment where the neoantigenic construct comprises more than one neoepitope peptide, each neoepitope peptide has the same amino acid sequence.

In embodiments where the neoantigenic construct may comprise more than one neoepitope peptide (e.g. one or more neoepitope peptides, two or more neoepitope peptides or three or more neoepitope peptides), and some of the neoepitope peptides have different amino acid sequences, or all of the neoepitope peptides have different amino acid sequences, the amino acid sequences may be different because:
the protein or peptide known or suspected to be expressed by a cancer cell in the subject are different; or
the protein or peptide known or suspected to be expressed by a cancer cell in the subject is the same, but the part of the protein or peptide known or suspected to be expressed by a cancer cell in the subject that the amino acid sequence of the neoepitope peptide corresponds to are different.

If the protein or peptide known or suspected to be expressed by a cancer cell in the subject is the same, but the part of the protein or peptide known or suspected to be expressed by a cancer cell in the subject that the amino acid sequence of the neoepitope peptide corresponds to are different, the parts may be different for one or more of the following reasons:
a longer part having the same at least one somatic mutated amino acid,
a shorter part having the same at least one somatic mutated amino acid, and/or
a part having the same at least one somatic mutated amino acid but the position of the mutated amino acid(s) is different in relation to the C- and N-terminus;
a different part of the same protein or peptide that has at least one different somatic mutated amino acid (for example, the protein or peptide has a frame shift mutation and the different parts are different parts of the frameshift mutated sequence of the protein or peptide).

In one preferred embodiment, the amino acid sequences are different because for each neoepitope peptide the protein or peptide known or suspected to be expressed by a cancer cell in the subject is different.

In one preferred embodiment, the amino acid sequences are different because the protein or peptide known or suspected to be expressed by a cancer cell in the subject is the same, but the part of the protein or peptide known or suspected to be expressed by a cancer cell in the subject that the amino acid sequence of the neoepitope peptide corresponds to are different because they are each different parts of a frameshift mutated sequence of the protein or peptide.

In embodiments of the invention wherein a neoantigenic construct comprises more than one neoepitope peptide (e.g. two or more neoepitope peptides or three or more neoepitope peptides), the neoepitope peptides may be directly linked, or linked via a spacer moiety.

In certain preferred embodiments wherein a neoantigenic construct comprises more than one neoepitope peptides (e.g. one or more neoepitope peptides, two or more neoepitope peptides or three or more neoepitope peptides), the neoepitope peptides are covalently linked.

A neoantigenic construct of the invention comprising more than one neoepitope peptide (e.g. two or more neoepitope peptides or three or more neoepitope peptides) may comprise neoepitope peptides that are directly linked and/or neoepitopes that are linked via a spacer moiety. If more than one spacer moiety is present in a neoantigenic construct, the spacer moieties of a neoantigenic construct may all be the same or they may be different.

In certain preferred embodiments wherein a neoantigenic construct comprises more than one neoepitope peptide (e.g. one or more neoepitope peptides, two or more neoepitope peptides or three or more neoepitope peptides), the neoepitope peptides are each linked via a spacer moiety.

A spacer moiety may be a short sequence of amino acids, for example 1 to 15 amino acids, preferably 1 to 10 amino acids, and more preferably 1 to 5 amino acids (for example, 1, 2, 3, 4 or 5 amino acids). The spacer moiety may comprise a random combination of amino acids; or a synthetic amino acid sequence, such as a polylysine, polyarginine, polyglycine, polyalanine or polyhistidine amino acid sequence. Preferably, the spacer moiety comprises the motif VVR and/or GGS.

The structure of the neoantigenic construct comprising two linked neoepitope peptides may be as follows:

-[1^{st} neoepitope peptide]-[spacer moiety]-[2^{st} neoepitope peptide].

The structure of the neoantigenic construct comprising three linked neoepitopes may be as follows:

-[1^{st} neoepitope peptide]-[spacer moiety]-[2^{nd} neoepitope peptide]-[spacer moiety]-[3^{rd} neoepitope peptide].

The structure of the neoantigenic construct comprising four linked neoepitopes may be as follows:

-[1^{st} neoepitope peptide]-[spacer moiety]-[2^{nd} neoepitope peptide]-[spacer moiety]-[3^{rd} neoepitope peptide]-[spacer moiety]-[4^{th} neoepitope peptide].

The structure of the neoantigenic construct comprising five linked neoepitopes may be as follows:

-[1^{st} neoepitope peptide]-[spacer moiety]-[2^{nd} neoepitope peptide]-[spacer moiety]-[3^{rd} neoepitope peptide]-[spacer moiety]-[4^{th} neoepitope peptide]-[spacer moiety]-[5^{th} neoepitope peptide].

The structure of the neoantigenic construct comprising n linked neoepitope peptide may be as follows:

-[1^{st} neoepitope peptide]-[spacer moiety]-[2^{nd} neoepitope peptide]-[spacer moiety]-[3^{rd} neoepitope peptide] . . . -[spacer moiety]-[n^{th} neoepitope peptide].

When the neoantigenic construct comprises more than one neoepitope peptide, the neoantigenic construct may consist of the neoepitope peptides and optionally any spacer moiety(s). Alternatively, when the neoantigenic construct comprises more than one neoepitope peptide, the neoantigenic construct may comprise the neoepitope peptides, optionally any spacer moiety(s) and a further amino acid sequence. Such a further amino acid sequence may, for example, be a random combination of amino acids (in particular random sequences having a high proportion of histidine and/or cysteine residues); or a synthetic amino acid sequence, such as a polylysine, polyarginine, polyglycine, polyalanine, polyhistidine or polycysteine amino acid sequence (and preferably a polyhistidine or polycysteine). Such a further amino acid sequence may be used, for example, as a linker and/or a spacer between the core of the phagocytosable particle and a neoepitope peptide of neoantigenic construct tightly associated to it, or used to tightly associate the neoantigenic construct to the phagocytosable particle. For example, metal chelates can bind proteins and peptides containing histidine or cysteine with great strength. Thus, cores with metal chelates can non-covalently bind to a neoantigenic construct comprising a polyhistidine or polycysteine synthetic amino acid sequence and/or a random combination of amino acids having a high proportion of histidine and/or cysteine residues as a further amino acid sequence. The neoantigen constructs described herein may also comprise an albumin-binding domain (ABD)

In embodiments of the invention, when the neoantigenic construct comprises one neoepitope peptide, the neoantigenic construct may consist of the neoepitope peptide. Alternatively, when the neoantigenic construct comprises one neoepitope peptide, the neoantigenic construct may comprise the neoepitope peptide and a further amino acid sequence. Such a further amino acid sequence may, for example, be a random combination of amino acids (in particular random sequences having a high proportion of histidine and/or cysteine residues); or a synthetic amino acid sequence, such as a polylysine, polyarginine, polyglycine, polyalanine, polyhistidine or polycysteine amino acid sequence (and preferably a polyhistidine or polycysteine). Such a further amino acid sequence may be used, for example, as a linker and/or a spacer between the core of the phagocytosable particle and a neoepitope peptide of neoantigenic construct tightly associated to it, or used to tightly associate the neoantigenic construct to the phagocytosable particle. For example, metal chelates can bind proteins and peptides containing histidine or cysteine with great strength. Thus, cores with metal chelates can non-covalently bind to a neoantigenic construct comprising a polyhistidine or polycysteine synthetic amino acid sequence and/or a random combination of amino acids having a high proportion of histidine and/or cysteine residues as a further amino acid sequence.

The length of a neoantigenic construct for use in the present invention depends on, for example, the number of neoepitope peptides in the neoantigenic construct, and the length of each neoepitope peptide in the neoantigenic construct, as well as the length of any spacer moieties that may be present if there is more than one neoepitope peptide, and the length of any further amino acids sequences that may be present. In certain preferred embodiments, the neoantigenic construct of the invention may have an amino acid sequence that is 3 to 300 amino acids, and preferably 10 to 250, more preferably 10 to 200 and more preferably 10 to 180 amino acids in length. For example, the neoantigenic construct of the invention may comprise 11 to 150 amino acids, 11 to 140 amino acids, 33 to 120 amino acids, 42 to 140 amino acids, 11 to 112 amino acids, 33 to 112 amino acids 2, 42 to 112 amino acids, 11 to 100 amino acids, 33 to 100 amino acids, 42 to 100 amino acids, 11 to 84 amino acids, 33 to 84 amino acids, 42 to 84 amino acids, 11 to 60 amino acids, 33 to 60 amino acids, or 42 to 60 amino acids, 11 to 50 amino acids, 33 to 50 amino acids, or 42 to 50 amino acids in length in length.

Neoantigenic constructs for use in the present invention can be prepared recombinantly (for example, in E. coli, mammalian cells or insect cells), synthetically (for example, using standard organic chemistry techniques, such as solution or solid phase peptide synthesis), or they may be prepared from polypeptides isolated from a native protein or peptide derived from an animal source, for example a human source. Preferably, neoantigenic constructs for use in the present invention are prepared recombinantly (for example, in E. coli, mammalian cells or insect cells). More preferably, neoantigenic constructs for use in the present invention are prepared recombinantly in E. coli.

The Phagocytosable Particle and the Core of the Phagocytosable Particle

A phagocytosable particle of the present invention is a particle able to be phagocytosed by cells of the immune system. It should be understood, however, that phagocytosable particles of the present invention may be internalised by cells of the immune system via different routes (e.g. pinocytosis, clathrin-mediated endocytosis and non-clathrin-mediated endocytosis). Preferably, the phagocytosable particles are phagocytosable by an APC, for example a monocyte, dendritic cell, B-cell or macrophage, or other cells that either phagocytose or internalise extracellular molecules, such as antigens, and present antigen-derived peptides on MHC class II and/or MHC class I molecules to CD4+ T-cells and/or CD8+ T-cells.

Antigens that are internalised into an APC by the phagocytic route are degraded in a non-uniform manner, which subsequently leads to a wider variety of antigen-derived peptides being presented by the APC. Without wishing to be bound by theory, it is believed that the phagocytosable particles for use in the present invention further improve the activation and expansion of anticancer T-cells because the neoantigenic constructs are phagocytosed by APCs, which subsequently leads to a wider variety of neoantigenic construct-derived peptides being presented by the APC and thus greater activation and expansion of anticancer T-cells.

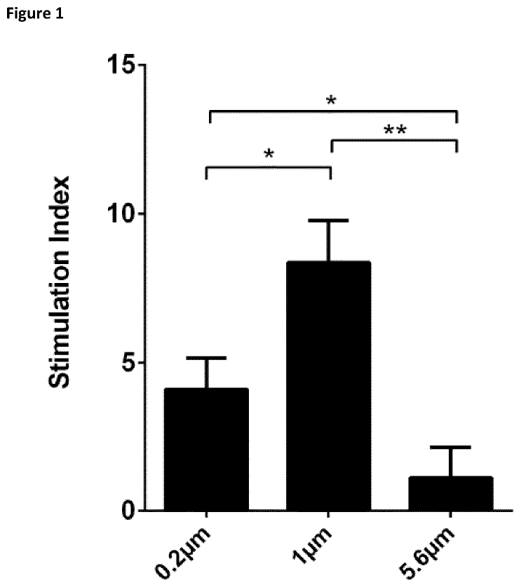

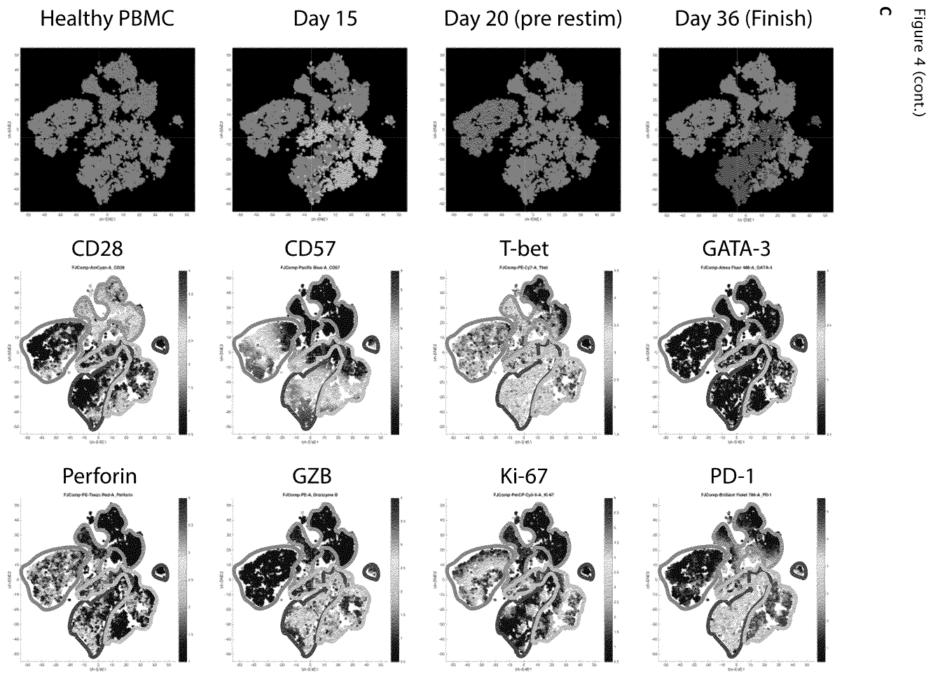

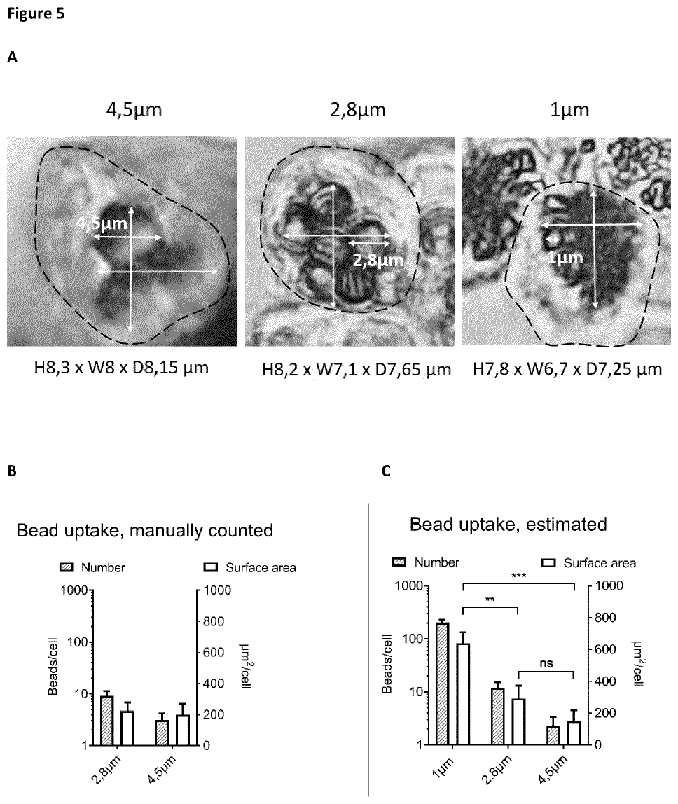

For a particle to be phagocytosed by a cell of the immune system, such as an APC, the particle needs to be within a size range suitable to allow for phagocytosis. For example, a particle that is too small may not trigger phagocytosis by a particular APC, or a particle that is too large may not be phagocytosable by a particular APC. Complete phagocytosis leads to good antigen degradation by APCs and subsequently good presentation to T-cells via the MHC class II pathway. The optimal size has been investigated by the current inventors (see Examples 3a and 3b, and FIGS. 1, 5A-C, and 6A-E).

A phagocytosable particle of the present invention comprises a core, and a neoantigenic construct tightly associated to the core. Thus, the size of the core needs to be within a range such that when the core is tightly associated to a neoantigenic construct(s), the core and the tightly associated neoantigenic construct(s) are phagocytosable by a cell of the immune system, and in particular an APC. It is preferred that the size of the core is within a range such that when the core is tightly associated to a neoantigenic construct(s), the core and the tightly associated neoantigenic construct(s) are small enough that more than one phagocytosable particle can enter the same APC by phagocytosis. Having more than one phagocytosed particle in an APC maximises presentation of neoepitope(s) on the cell surface via the MHC class II pathway. Furthermore, it allows particles having different neoantigenic constructs (in particular neoantigenic constructs comprising different neoepitope peptides) to enter an APC, which means that the APC can present different neoepitopes from several particles in different phagosomes at the same.

As such, in one preferred embodiment, the core has a largest dimension of less than 6 μm, less than 5.6 μm, less than 4 μm, less than 3 μm, less than 2.5 μm, less than 2 μm or less than 1.5 μm. More preferably the core has a largest dimension of less 1.5 μm. In another preferred embodiment, the core may have a largest dimension of greater than 0.001 μm, greater than 0.005 μm, greater than 0.01 μm, greater than 0.05 μm, greater than 0.1 μm, greater than 0.2 μm or greater than 0.5 μm. More preferably the core has a largest dimension of greater than 0.5 μm.

In one especially preferred embodiment, the core has a largest dimension in the range of 0.1 to 6 μm, for example 0.1 to 5.6 μm, 0.2 to 5.6 μm, 0.5 to 5.6 μm, 0.1 to 4 μm or 0.5 to 4 μm. More preferably, the core has a largest dimension in the range of 0.1 to 3 μm, for example, 0.5 to 3 μm, 0.2 to 2.5 μm, 0.5 to 2.5 μm, 0.2 to 2 μm, 0.5 to 2 μm or 1 to 2 μm. Even more preferably, the core has a largest dimension of about 1 μm, about 1.5 μm or about 2 μm. In a very preferred embodiment of the invention, the core is about 1 μm.

The core of the phagocytosable particle of the invention takes the form of any three-dimensional shape, for example any regular or irregular three-dimensional shape. Preferably, the phagocytosable particle is substantially spherical, in which case the dimensions of the phagocytosable particle refers to diameter.

The core of a phagocytosable particle may comprise a polymer, glass, ceramic material (e.g. the core may be a polymer particle, a glass particle or a ceramic particle). The material of the core may be a biodegradable and/or biocompatible material (e.g. the particle may be a biodegradable and/or biocompatible particle).

Preferably the core comprises a polymer (for example, the core is a polymer particle). If the core comprises a polymer, it may be selected from the group consisting of a synthetic aromatic polymer (such as polystyrene e.g. the core is a polystyrene particle), a synthetic non-aromatic polymer (such as polyethylene, polylactic acid, poly(lactic-co-glycolic acid) and polycaprolactone, e.g. the core is a polyethylene particle, polylactic acid particle, poly(lactic-co-glycolic acid) particle or polycaprolactone particle), a naturally occurring polymer (such as collagen, gelatine, proteins (e.g. virus-like particles), lipids or albumin, e.g. the core is a collagen particle, gelatine particle or albumin particle), a polymeric carbohydrate molecule (such as a polysaccharide, for example agarose, alginate, chitosan or zymosan e.g. the core is an agarose particle, alginate particle, chitosan particle or zymosan particle).

In one preferred embodiment the core comprises polystyrene or polyethylene, and more preferably comprises polystyrene (e.g. the core is a polystyrene particle). Such polymers are biocompatible.

The present inventors have found that polystyrene particles are a particularly useful core for a phagocytosable particle of the present invention because they are nontoxic and are widely commercially available in various sizes and in various functionalisable forms. Furthermore, the present inventors have found phagocytosable particles comprising a polystyrene core, such as a polystyrene particle, are able to withstand stringent sterilisation procedures to prepare the particles for administration to a subject. Such sterilisation procedures may include repeated washes with acid or alkali solutions and/or exposure to high temperatures.

In one very preferred embodiment of the invention, the core is a polystyrene particle with a largest dimension of less than 6 μm, preferably from about 1 μm to about 3 μm, and more preferably about 1 μm. Phagocytosable particles comprising a polystyrene particle core with a dimension of about 1 μm to about 3 μm, and especially about 1 μm, are efficiently phagocytosed by APCs and are also able to withstand stringent sterilisation procedures to remove pathogens (e.g. bacteria, fungus and viruses) and antigenic contaminants, such as pyrogens (e.g. endotoxins), which may be associated to the core or neoantigenic construct.

In one embodiment of the invention, the core has magnetic properties. For example, the core may have paramagnetic or superparamagnetic properties. Preferably, the core has superparamagnetic properties.

An example of a superparamagnetic core suitable for use with the invention are Dynabeads™ (Invitrogen). Dynabeads™ are available in various functionalisable forms, for example Dynabeads M-270 Carboxylic acid, Dynabeads M-270 Amine, and Dynabeads MyOne Carboxylic acid. Dynabeads™ are monosized superparamagnetic particles, which are composed of highly cross-linked polystyrene with evenly distributed magnetic material. The magnetic material may be iron oxide. Other examples of magnetic cores, in particular superparamagnetic cores, include Encapsulated Carboxylated Estapor® SuperParamagnetic Microspheres (Merck Chimie S.A.S.) and Sera-Mag SpeedBeads (hydrophilic) Carboxylate-Modified Magnetic particles (GE Healthcare UK Limited). Encapsulated Carboxylated Estapor® SuperParamagnetic Microspheres are made of a core-shell structure which encapsulates an iron oxide core.

A phagocytosable particle of the present invention comprises a neoantigenic construct tightly associated to the core. A neoantigenic construct may be tightly associated to a core using a variety of means. For example, the neoantigenic construct may be attached to a core by a covalent bond, for example an amide bond between an amine group or a carboxylic acid group of the neoantigenic construct and a carboxylic acid group or an amine group on the surface of the core. Alternatively, a neoantigenic construct may be linked to a core via a metal chelate. For example, cores linked with a metal chelating ligand, such as iminodiacetic acid can bind metal ions such as Cu²⁺, Zn²⁺, Ca²⁺, Co²⁺ or Fe³⁺. These metal chelates can in turn bind proteins and peptides containing for example histidine or cysteine with great strength. Thus, cores with metal chelates can non-covalently bind to a neoantigenic construct. Preferably, the neoantigenic construct is covalently attached to the core. One example of associating the neoantigenic construct to the core is shown in Example 1.

A phagocytosable particle of the present invention comprises a core, and a neoantigenic construct tightly associated to the core. A phagocytosable particle of the invention may comprise one or more neoantigenic constructs associated to the core. For example, a phagocytosable particle of the invention may comprise 1 to 3 million neoantigenic constructs, preferably 1 to 2 million neoantigenic constructs, and more preferably 1 to 1 million neoantigenic constructs, for example 1 to 800,000, 1 to 500,000, 1 to 100,000, 1 to 10,000, 1 to 1000, 1 to 100, or 1 to 10 neoantigenic constructs; or for example 10 to 1 million, 100 to 1 million, 1000 to 1 million, 10,000 to 1 million, 100,000 to 1 million, or 500,000 to 1 million. Preferably a phagocytosable particle of the invention may comprise 500,000 to 1 million neoantigenic constructs.

In preferred embodiments, to maximise the delivery of neoantigenic construct into an APC (which can then be cleaved from the phagocytosable particle and processed by an APC, thus resulting in the presentation of a wide variety of neoepitope-derived peptides on the surface of the APCs), a phagocytosable particle of the invention may comprise more than one (i.e. two or more, for example two to 3 million) neoantigenic constructs associated to the core. For example, phagocytosable particle of the invention may comprise 2 to 1 million neoantigenic constructs tightly associated to a core (for example 2 to 800,000, 2 to 500,000, 2 to 100,000, 2 to 10,000, 2 to 1000, 2 to 100, or 2 to 10 neoantigenic constructs tightly associated to a core). Preferably, a phagocytosable particle of the invention comprises 10 or more neoantigenic constructs tightly associated to a core, such as 10 to 1 million neoantigenic constructs tightly associated to a core (for example 10 to 800,000, 10 to 500,000, 10 to 100,000, 10 to 10,000, 10 to 1000, or 10 to 100 neoantigenic constructs tightly associated to a core). More preferably a phagocytosable particle of the invention comprises 100 or more neoantigenic constructs tightly associated to a core, such as 100 to 1 million neoantigenic constructs tightly associated to a core (for example 100 to 800,000, 100 to 500,000, 100 to 100,000, 100 to 10,000, or 100 to 1000 neoantigenic constructs tightly associated to a core). In certain embodiments a phagocytosable particle of the invention comprises 1000 or more genic constructs tightly associated to a core, such as 1000 to 1 million neoantigenic constructs tightly associated to a core (for example 1000 to 800,000, 1000 to 500,000, 1000 to 100,000, or 1000 to 10,000 neoantigenic constructs tightly associated to a core). In certain embodiments a phagocytosable particle of the invention comprises 10,000 or more neoantigenic constructs tightly associated to a core, such as 10,000 to 1 million neoantigenic constructs tightly associated to a core (for example 10,000 to 800,000, 10,000 to 500,000, or 10,000 to 100,000 neoantigenic constructs tightly associated to a core). In certain embodiments a phagocytosable particle of the invention comprises 100,000 or more neoantigenic constructs tightly associated to a core, such as 100,000 to 1 million neoantigenic constructs tightly associated to a core (for example 100,000 to 800,000 or 100,000 to 500,000 neoantigenic constructs tightly associated to a core). In one very preferred embodiment a phagocytosable particle of the invention comprises 500,000 or more neoantigenic constructs tightly associated to a core, such as 500,000 to 1 million neoantigenic constructs tightly associated to a core, or 500,000 to 2 million neoantigenic constructs tightly associated to a core, or 500,000 to 3 million neoantigenic constructs tightly associated to a core. In another embodiment a phagocytosable particle of the invention may comprise more than 1 million neoantigenic constructs tightly associated to a core, for example 1 million to 3 million, or 1 million to 2 million neoantigenic constructs tightly associated to a core.

In embodiments of the invention, where a phagocytosable particle of the invention may comprise more than 1 neoantigenic construct associated to the core (for example 2 or more, 10 or more, 100 or more, 1000 or more, 10,000 or more, 100,000 or more, or 500,000 or more neoantigenic constructs associated to the core), the neoantigenic constructs associated to the core may be the same, or may be different (i.e. some or all of the neoantigenic constructs associated to the core may be different). They may be different by comprising different neoepitope peptide sequences or they may be different by comprising a different combination of neoepitope peptides. They may alternatively, or additionally, be different by comprising one or more different spacer moieties or further amino acid sequences, if such moieties and sequences are present. Neoantigenic constructs that are different may be referred to as “different types” of neoantigenic construct. Neoantigenic constructs that are the same may be referred to as the “same type” of neoantigenic construct. Cancer cells often induce multiple amino acid mutations in multiple proteins or peptides expressed by the cancer cell. The present inventors have found that phagocytosable particles comprising two or more different types of neoantigenic constructs are able to deliver a wide variety of neoepitopes into an APC and thus increase the variety of neoepitope-derived peptides that are presented by the APC. The present inventors have found that this significantly improves the activation and expansion of anticancer T-cells that are able to target cancer cells.

A phagocytosable particle comprising more than 1 neoantigenic construct associated to the core (for example 2 or more, 10 or more, 100 or more, 1000 or more, 10,000 or more, 100,000 or more, or 500,000 or more neoantigenic construct associated to the core) of the invention can comprise one type of neoantigenic construct tightly associated to a core (i.e. all the neoantigenic construct tightly associated to the core are the same). In one embodiment of the invention, a phagocytosable particle comprises 100,000 to 1 million neoantigenic constructs tightly associated to a core, wherein the 100,000 to 1 million neoantigenic constructs are the same type of neoantigenic construct.

In an alternative embodiment of the invention, a phagocytosable particle comprising more than 1 neoantigenic construct associated to the core (for example 2 or more, 10 or more, 100 or more, 1000 or more, 10,000 or more, 100,000 or more, or 500,000 or more neoantigenic constructs associated to the core) can comprise two different neoantigenic construct types tightly associated to a core. In an another embodiment of the invention, a phagocytosable particle comprising more than 10 neoantigenic constructs associated to the core (for example 100 or more, 1000 or more, 10,000 or more, 100,000 or more, or 500,000 or more neoantigenic constructs associated to the core) can comprise two or more different neoantigenic construct types tightly associated to a core. For example such a phagocytosable particle may comprise 2 to 10 different neoantigenic construct types tightly associated to a core (for example 2, 3, 4, 5, 6, 7, 8, 9 or 10). Preferably, such a phagocytosable particle may comprises 2 to 6 different neoantigenic constructs types (for example 2, 3, 4, 5 or 6). In one embodiment of the invention, a phagocytosable particle comprises 100,000 to 1 million neoantigenic constructs tightly associated to a core, wherein the 100,000 to 1 million neoantigenic constructs comprise two or more different neoantigenic construct types. For example 2 to 6 different neoantigenic construct types (for example 2, 3, 4, 5 or 6).

In one embodiment of the invention, the phagocytosable particle further comprises an adjuvant tightly associated to the core. The term “adjuvant” as used herein is to be understood as any substance that enhances an immune response towards an antigen. Particular examples of adjuvants include dsRNA analogues, such as polyinosinic:polycytidylic acid, Incomplete Freund's Adjuvant, cytokines (for example, IL-2, IL-4, IL-17 and IL-15), CD40, keyhole limpet hemocyanin, Toll-like receptors, CpG oligodeoxynucleotides, saponins, colloidal alum, and analogues of lipid A of lipopolysaccharide. Adjuvants may be tightly associated to the core in the same manner as that described herein for tightly associating a neoantigenic construct to a core.

Sterilisation of the Phagocytosable Particles

The present inventors have advantageously found that phagocytosable particles comprising a core and a neoantigenic construct tightly associated to the core, may be efficiently washed and sterilised before administration to a subject. This is particularly advantageous because the washed and sterilised phagocytosable particles comprise lower levels of pathogens (e.g. bacteria, fungus and viruses) and contaminants, such as endotoxins (e.g. lipopolysaccharides) and other antigenic contaminants. Such contaminants can elicit non-specific immune responses in the subject. Washing and sterilising phagocytosable particles of the invention before administration therefore improves their safety and efficacy.

In one embodiment of the invention, the phagocytosable particle comprises a magnetic core, for example a paramagnetic or superparamagnetic core. A phagocytosable particle comprising a magnetic core, can be collected and/or held in place by a magnet. It is also possible to perform a wash by other means, such as by holding the phagocytosable particles (whether paramagnetic or not) in a column, or sedimenting the particles by gravity or by centrifugation.

The particular manner of the wash is not critical in the context of the present invention. For instance, the wash may involve subjecting a phagocytosable particle to a high pH, to a low pH, to a high temperature, to a sterilising/denaturing agent or a combination thereof

The wash may involve subjecting the phagocytosable particle to alkali, preferably a strong alkali, for example at least 0.1M, 0.5M, 1M, 2M, 3M, 4M, 5M, 6M, 7M or 8M alkali. Preferably, the wash may involve subjecting the phagocytosable particle to at least 1M sodium hydroxide (NaOH), for example at least 2M NaOH. Preferably, the wash involves subjecting the phagocytosable particle to a high pH of at least 13.0, more preferably at least 14.0, most preferably at least 14.3. Other alkalis that may be used include, but are not limited to: lithium hydroxide (LiOH), potassium hydroxide, (KOH), rubidium hydroxide (RbOH), cesium hydroxide (CsOH), magnesium hydroxide (Mg(OH)₂), calcium hydroxide (Ca(OH)₂), strontium hydroxide (Sr(OH)₂), and barium hydroxide (Ba(OH)₂). Preferably, the wash involves subjecting the phagocytosable particle to a high pH of at least 13.0, more preferably at least 14.0, most preferably at least 14.3.

The wash may also involve subjecting the phagocytosable particle to an acid, preferably a strong acid, for example at least 0.1M, 0.5M, 1M, 2M, 3M, 4M, 5M, 6M, 7M or 8M acid. Preferably, the wash may involve subjecting the phagocytosable particle to at least 1M hydrochloric acid (HCl), for example at least 2M HCl. Other acids that may be used include, but are not limited to: hydroiodic acid (HI), hydrobromic acid (HBr), perchloric acid (HClO₄), nitric acid (HNO₃) and sulfuric acid (H₂SO₄).

The wash may also involve subjecting the phagocytosable particle to further sterilising/denaturing agents, such as urea and/or guanidine-HCl.

Preferably, the wash results in the phagocytosable particle being aseptic and/or sterile. More preferably, the wash results in the phagocytosable particle being sterile. Aspetic as defined herein is being free from disease-causing microorganisms and viruses. Sterile is defined herein as being free from all biological contaminants.

Preferably, the wash also removes antigenic contaminants such as pyrogens (e.g. endotoxins) from the phagocytosable particle. Preferably, the wash provides the phagocytosable particle with an endotoxin contamination of less than 100 pg/ml, preferably less than 50 pg/ml, more preferably less than 25 pg/ml and most preferably less than 10 pg/ml.

Thus, in a preferred embodiment of the invention the phagocytosable particle is sterile and has an endotoxin contamination of less than 100 pg/ml.

A particular advantage of the wash is that the conditions may be selected such that the phagocytosable particle is both sterilized and denatured in a single step. In particular, a high pH wash (e.g. pH>14) can conveniently, simultaneously and quickly, sterilise the phagocytosable particle and eliminate a sufficient quantity of endotoxin and other antigenic contaminants.

The wash may comprise a single wash or several repeated washes, such as 2, 3, 4 or 5 washes. In addition, or alternatively, the phagocytosable particle may be subjected to a high temperature, such as at least 90° C., preferably at least 92° C., more preferably at least 95° C., for example at least 100° C. or at least 110° C.

The present inventors have advantageously found that when a neoantigenic construct is associated to a core by a covalent bond, the phagocytosable particle can withstand stringent sterilisation and washing procedures that reduce the amount of antigenic contaminants, such as pyrogens (e.g. endotoxins), that may be bound to a neoantigenic construct or core. This means that the phagocytosable particles described herein are especially suitable for use in the treatment or prophylaxis of cancer.

Injectable Compositions

The present invention provides an injectable composition comprising a phagocytosable particle of the invention.

An injectable composition of the invention comprising a phagocytosable particle as described herein may comprise one or more phagocytosable particles. Preferably, it comprises more than one particle. In such embodiments, the phagocytosable particles may be the same, or they may be different. Phagocytosable particles may be different due to the core and/or may be different due to comprising different types of neoantigenic construct(s) tightly associated to the core. Preferably, in a composition comprising phagocytosable particles of the invention in which the phagocytosable particles are different, the phagocytosable particles have the same core and are different due to the particles comprising different types of neoantigenic constructs tightly associated to the core.

Phagocytosable particles having different cores (e.g. cores having different sizes and/or comprising different materials/polymers as described herein) are referred to herein as phagocytosable particles of a “different set”. Phagocytosable particles having the same core (e.g. cores having the same size and comprising the same materials/polymer) may be referred to herein as phagocytosable particles of the “same set”.

Phagocytosable particles that have the same core (i.e. they are of the same phagocytosable particle set), but that are different due to having different types of neoantigenic construct tightly associated to the core, are referred to herein as phagocytosable particles of “different groups”. Phagocytosable particles that have the same core and the same type of neoantigenic constructs tightly associated to the core are referred to herein as phagocytosable particles of the “same group”.

In one embodiment, an injectable composition of the invention comprises one phagocytosable particle set which consists of one phagocytosable particle group (i.e. all of the phagocytosable particles in the composition are the same). Alternatively, an injectable composition of the invention can comprise one phagocytosable particle set which comprises two or more different groups of phagocytosable particle (for example 2, 3, 4, 5, 6, 7, 8, 9, 10, 12, 15, 20, 25, 30, 40 or 50, or more, phagocytosable particle groups). In one embodiment, an injectable composition of the invention can comprise one phagocytosable particle set which comprises 2 to 50 different groups of phagocytosable particle (for example 2, 3, 4, 5, 6, 7, 8, 9, 10, 12, 15, 18, 20, 25, 30, 40 or 50 phagocytosable particle groups). In one embodiment, an injectable composition of the invention can comprise one phagocytosable particle set which comprises 2 to 30 different groups of phagocytosable particle (for example 2, 3, 4, 5, 6, 7, 8, 9, 10, 12, 15, 18, 20, 25, or 30 phagocytosable particle groups). In another embodiment, an injectable composition of the invention can comprise one phagocytosable particle set which comprises 2 to 20 different groups of phagocytosable particle (for example 2, 3, 4, 5, 6, 7, 8, 9, 10, 12, 15, 18 or 20 phagocytosable particle groups). In another embodiment, an injectable composition of the invention can comprise one phagocytosable particle set which comprises 2 to 15 different groups of phagocytosable particle (for example 2, 3, 4, 5, 6, 7, 8, 9, 10, 12, or 15 phagocytosable particle groups). In another embodiment, an injectable composition of the invention can comprise one phagocytosable particle set which comprises 2 to 10 different groups of phagocytosable particle (for example 2, 3, 4, 5, 6, 7, 8, 9, or 10 phagocytosable particle groups). In another embodiment, an injectable composition of the invention can comprise one phagocytosable particle set which comprises 2 to 8 different groups of phagocytosable particle (for example 2, 3, 4, 5, 6, 7, or 8 phagocytosable particle groups). In another embodiment, an injectable composition of the invention can comprise one phagocytosable particle set which comprises 2 to 6 different groups of phagocytosable particle (for example 2, 3, 4, 5, or 6 phagocytosable particle groups).

In one embodiment, an injectable composition of the invention can comprise two or more different phagocytosable particle sets (i.e. each set having different cores, for example each set having cores of different sizes and/or comprising different materials as described herein) and each set can consist of one phagocytosable particle group. For example, the injectable composition of the invention can comprise 2, 3, 4 or 5 phagocytosable particle sets and each set can consist of one phagocytosable particle group. Preferably, the injectable composition of the invention can comprise 2 or 3 phagocytosable particle sets and each set consists of one phagocytosable particle group.

In another embodiment, an injectable composition of the invention can comprise two or more different phagocytosable particle sets (for example 2, 3 or 4 phagocytosable particle sets) and each set can comprise of two or more different phagocytosable particle group (for example 2, 3, 4, 5, 6, 7, 8, 9, 10, 12, 15, 20, 25 or 30 phagocytosable particle groups). In one embodiment, an injectable composition of the invention can comprise two or more different phagocytosable particle sets (for example 2, 3 or 4 phagocytosable particle sets) and each set can comprise 2 to 30 different groups of phagocytosable particle (for example 2, 3, 4, 5, 6, 7, 8, 9, 10, 12, 15, 18, 20, 25 or 30 phagocytosable particle group). In another embodiment, an injectable composition of the invention can comprise two or more different phagocytosable particle sets (for example 2, 3 or 4 phagocytosable particle sets) and each set can comprise 2 to 20 different groups of phagocytosable particle (for example 2, 3, 4, 5, 6, 7, 8, 9, 10, 12, 15, 18 or 20 phagocytosable particle groups). In another embodiment, an injectable composition of the invention can comprise two or more different phagocytosable particle sets (for example 2, 3 or 4 phagocytosable particle sets) and each set can comprise 2 to 15 different groups of phagocytosable particle (for example 2, 3, 4, 5, 6, 7, 8, 9, 10, 12, or 15 phagocytosable particle groups). In another embodiment, an injectable composition of the invention can comprise two or more different phagocytosable particle sets (for example 2, 3 or 4 phagocytosable particle sets) and each set can comprise 2 to 10 different groups of phagocytosable particle (for example 2, 3, 4, 5, 6, 7, 8, 9, or 10 phagocytosable particle groups). In another embodiment, an injectable composition of the invention can comprise two or more different phagocytosable particle sets (for example 2, 3 or 4 phagocytosable particle sets) and each set can comprise 2 to 8 different groups of phagocytosable particle (for example 2, 3, 4, 5, 6, 7, or 8 phagocytosable particle groups). In another embodiment, an injectable composition of the invention can comprise two or more different phagocytosable particle sets (for example 2, 3 or 4 phagocytosable particle sets) and each set can comprise 2 to 6 different groups of phagocytosable particle (for example 2, 3, 4, 5, or 6 phagocytosable particle groups).

For the avoidance of doubt, in embodiments having more than one group of phagocytosable particle and more than one set of phagocytosable particle, each group of a phagocytosable particle set is independent from each group of another phagocytosable particle set. Thus, a group from one phagocytosable particle set can have the same neoantigenic construct type as a group from another phagocytosable particle set. Alternatively, a group from one phagocytosable particle set can have neoantigenic constructs that are a different type to those of a group from another phagocytosable particle set.

Cancer cells often induce multiple amino acid mutations in multiple proteins or peptides expressed by the cancer cell. The administration of an injectable composition comprising two or more phagocytosable particle groups allows for delivery of a wide variety of neoepitopes into an APC and thus increases the variety of neoepitope-derived peptides that are presented by the APC. An increased variety of neoepitope-derived peptides presented by an APC can significantly improve the activation and expansion of anticancer T-cells.

Pharmaceutically acceptable injectable formulations useful according to the invention include those suitable for parenteral (including subcutaneous, intradermal, intramuscular, intravenous (bolus or infusion), intraarticular, and intralymphatic) administration. The most suitable route may depend upon, for example, the condition and disorder of the recipient. Preferably, the pharmaceutical composition is suitable for intravenous and/or intralymphatic administration. Thus, the present invention provides an injectable composition comprising a phagocytosable particle of the invention.

Preferably, the injectable composition of the invention is an injectable pharmaceutical composition. The injectable composition of the invention includes compositions suitable for subcutaneous, intradermal, intramuscular, intravenous (bolus or infusion), intratumorally, intraarticular and intralymphatic administration, although the most suitable route may depend upon, for example, the type of cancer or tumour present in the subject. Preferably, the injectable composition is suitable for intravenous and/or intralymphatic administration.

Pharmaceutically acceptable injectable formulations of the invention and the injectable compositions of the invention may include aqueous and non-aqueous sterile injection solutions (e.g. saline, such as PBS) which may contain anti-oxidants, buffers (e.g. sodium phosphate, potassium phosphate, TRIS and TEA), bacteriostats, surfactants (e.g. poloxamers, polysorbates, CHAPS and Titon X-100), and solutes which render the composition isotonic with the blood of the intended recipient; and aqueous and non-aqueous sterile suspensions which may include suspending agents and thickening agents.

The compositions may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use. Extemporaneous injection solutions and suspensions for parenteral administration include injectable solutions or suspensions which can contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor.

Pharmaceutically acceptable formulations of the invention and injectable compositions of the invention may further comprise an adjuvant. The term “adjuvant” as used herein is to be understood as any substance that enhances an immune response towards an antigen. Examples of adjuvants for use in the present invention include dsRNA analogues, such as polyinosinic:polycytidylic acid, Incomplete Freund's Adjuvant, cytokines (for example, interleukins), CD40, keyhole limpet hemocyanin, Toll-like receptors, CpG oligodeoxynucleotides, saponins, colloidal alum, and analogues of lipid A of lipopolysaccharide. Thus, an injectable composition of the invention may comprise dsRNA analogues, such as polyinosinic:polycytidylic acid, Incomplete Freund's Adjuvant, cytokines (for example, IL-2, IL-4, IL-17 and IL-15), CD40, keyhole limpet hemocyanin, Toll-like receptors, CpG oligodeoxynucleotides, saponins, colloidal alum, and analogues of lipid A of lipopolysaccharide.

Preferred unit dosages of pharmaceutically acceptable formulations of the invention and injectable compositions of the invention are those containing a therapeutic dose (i.e. a dose suitable to elicit a primary immune response to a cancer) or a booster dose (i.e. a dose suitable to induce a secondary immune response to a cancer), or an appropriate fraction thereof, of phagocytosable particles. A unit dosage of phagocytosable particles may be 1 μg to 4000 μg, 10 μg to 3000 μg or 10 μg to 2000 μg. For example, the unit dosage may be 10 μg to 1000 μg, 10 μg to 750 μg, 10 μg to 500 μg, 20 to 400 μg, 25 μg to 300 μg, or 30 μg to 200 μg, 50 μg to 1000 μg, 50 μg to 750 μg, 50 μg to 500 μg, 50 μg to 400 μg, 50 μg to 300 μg or 50 μg to 200 μg, 100 μg to 1000 μg, 100 μg to 750 μg, 100 μg to 500 μg, 100 μg to 400 μg, 100 μg to 300 μg or 100 μg to 200 μg, 200 μg to 1000 μg, 200 μg to 750 μg, 200 μg to 500 μg, 200 μg to 400 μg, 200 μg to 300 μg, 400 μg to 1000 μg, 400 μg to 750 μg, 400 μg to 500 μg, 500 μg to 1000 μg, 500 μg to 750 μg, or 750 μg to 1000 μg. For example, a unit dosage of phagocytosable particles may be 1, 10, 50, 100, 200, 250, 300, 400, 500, 600, 700, 750, 800, 900, 1000, 1500, 2000, 3000 or 4000 μg. Preferably, the unit dosage is 100 to 750 μg, more preferably, 200 to 750 μg, more preferably 300 to 750 μg, more preferably 400 to 750 μg, more preferably 500 to 750 μg, more preferably, 600 to 750 μg, even more preferably 650 to 750 μg. For example, a unit dosage of phagocytosable particles may be, 100, 200, 300, 400, 500, 600, 700 or 750 μg.

It should be understood that in addition to the ingredients particularly mentioned above, the pharmaceutically acceptable formulations of the invention and injectable compositions of the invention may include other agents conventional in the art having regard to the type of composition in question.

Whilst the phagocytosable particles of the invention for use in the various embodiments of the present invention may be used as the sole active ingredient, it is also possible for the phagocytosable particles to be used in combination with one or more further active agents. Thus, the invention also provides a phagocytosable particle for use in the treatment or prophylaxis of cancer in a subject, or for use in methods of treatment or prophylaxis of cancer in a subject, according to the invention together with a further active agent, for simultaneous, sequential or separate administration. Such further active agents may be agents useful in the treatment of cancer, or other pharmaceutically active materials, but are preferably agents known for the treatment of cancer. Such agents are known in the art. Examples of further active agents include alkylators, antimetabolites, anti-tumour antibiotics, histone deacetylase inhibitors, immunomodulatory drugs, microtubule interactive drugs, protein kinase inhibitors, steroids, topoisomerase inhibitors, cell cycle inhibitors, and angiogenesis inhibitors.

Thus, a phagocytosable particle of the present invention for use in the treatment or prophylaxis of cancer in a subject, or for use in methods of treatment or prophylaxis of cancer in a subject of the present invention, may be administered together with one or more compounds known for the treatment or prophylaxis of cancer, for example one or more alkylator, antimetabolite, anti-tumour antibiotic, histone deacetylase inhibitor, immunomodulatory drug, microtubule interactive drugs, protein kinase inhibitor, steroid, topoisomerase inhibitor cell cycle inhibitor, or angiogenesis inhibitor.

When used in a combination, the precise dosage of the further active agent(s) will vary with the dosing schedule, the oral potency of the particular agent chosen, the age, size, sex and condition of the subject (typically mammal or human), the nature and severity of the cancer, and other relevant medical and physical factors. Thus, a precise therapeutic dose or booster dose cannot be specified in advance and can be readily determined by the caregiver or clinician. An appropriate amount can be determined by routine experimentation from animal models and human clinical studies. For humans, a therapeutic or booster dose will be known or otherwise be determined by one of ordinary skill in the art.

The individual components of such combinations can be administered separately at different times during the course of therapy or concurrently in divided or single combination forms. The present invention is therefore to be understood as embracing all such regimes of simultaneous or alternating treatment.

The above further active agent(s), when employed in combination with compounds useful in the invention, may be used, for example, in those amounts indicated in the Physicians' Desk Reference (PDR) or as otherwise determined by one of ordinary skill in the art.

Treatments

The present invention provides a phagocytosable particle for use in the treatment or prophylaxis of cancer in a subject, wherein the phagocytosable particle comprises a core and a neoantigenic construct tightly associated to the core, and wherein the neoantigenic construct comprises a neoepitope peptide having an amino acid sequence corresponding to an amino acid sequence of a part of a protein or peptide known or suspected to be expressed by a cancer cell in the subject, wherein the part of the protein or peptide has at least one somatic mutated amino acid. The present invention also provides methods of treating or preventing cancer in a subject comprising the step of administering to the subject a phagocytosable particle of the invention. The present invention also provides a use of a phagocytosable particle of the invention for the manufacture of a medicament for the treatment or prophylaxis of cancer.

The present inventors have found that phagocytosable particles used in the invention are surprisingly effective at eliciting a robust anticancer immune response towards a cancer cell in a subject. On a general level, immune responses can be categorised as either an innate immune response or an adaptive immune response. An innate immune response is an immune response that is not intrinsically affected by prior contact with an antigen. Such a response is often characterised by the activation and expansion of naïve T-cells and B-cells. In contrast, an adaptive immune response is an immune response that requires prior contact with an antigen. The adaptive immune response generally follows shortly after an innate immune response, and ultimately leads to immunological memory towards an antigen. When the immune system comes into contact with an antigen for the first time, the immune response that is elicited (innate and adaptive responses) is often referred to as a “primary immune response”. Later activation of the memory T-cells and B-cells by the same antigen results in a rapid and specific immune response towards the antigen, this rapid and specific immune response towards the antigen is often referred to as a “secondary immune response”.

The present investors have surprisingly found that the phagocytosable particles of the present invention elicit a robust anticancer immune response by activating both the innate and adaptive immune response in a subject.

The immune response induced by a phagocytosable particle used in the present invention may involve the phagocytosis of the phagocytosable particle by an antigen presenting cell (APC). APCs are typically dendritic cells (DCs), B-cells or macrophages, or cells that either phagocytose or internalise extra-cellular organisms or proteins, i.e. antigens, and after processing present antigen-derived peptides on MHC class II and/or MHC class I to CD4+ T-cells and/or CD8+ T-cells. In blood, monocytes are the most abundant APCs, for example dendritic cells, macrophages and B cells.

The immune response induced by a phagocytosable particle used in the present invention may also induce activation and expansion of naïve or memory T-cells. For example, the phagocytosable particle of the invention may induce the activation and expansion of CD4+ T-cells (or T-helper cells or CD4+ helper T-cells) and/or CD8+ T-cells (or cytotoxic T-cells). CD4+ T-cells are cells that orchestrate immune responses through cytokine secretion. They can both suppress or potentiate other immune cells, such as stimulate antibody class switching of B-cells, stimulate activation and expansion of cytotoxic T-cells or potentiate phagocytes. They get activated by antigen presentation via MHC class II on APCs and they express a T-cell receptor (TCR) specific for a stretch of approximately 15 amino acids (a so-called T-cell epitope) within a particular antigen. CD8+ T-cells (or cytotoxic T-cells) are cells that kill tumour cells, infected cells or cells otherwise damaged. Unlike CD4+ T-cells they do not need APCs for activation. Their T-cell receptor recognizes antigen derived peptides (approximately 7-10, for example 8, amino acids long) presented by MHC class I, a protein expressed on all nucleated cells.

The treatments of the invention may be used to treat or prevent any form of cancer, for example a solid cancer, a metastatic solid cancer or a hematologic malignancy.

A “solid cancer” herein is, for example, an abnormal mass of tissue that originates in an organ. The solid cancer may be malignant. Different types of solid cancers are named for the type of cells that form them. Types of solid cancer include sarcomas, carcinomas, and lymphomas. Examples of solid cancers include adrenal cancer, anal cancer, anaplastic large cell lymphoma, angioimmunoblastic T-cell lymphoma, B-cell lymphoma, bile duct cancer, urinary bladder cancer, brain/CNS tumours, breast cancer, cervical cancer, colon cancer, endometrial cancer, oesophagus cancer, ewing family of tumours, eye cancer, gallbladder cancer, gastrointestinal carcinoid tumours, gastrointestinal stromal tumour (gist), gestational trophoblastic disease, hepatosplenic T-cell lymphoma, Hodgkin's lymphoma, intravascular large B-cell lymphoma, kidney cancer, laryngeal and hypopharyngeal cancer, liver cancer, lung cancer (non-small cell and small cell), lung carcinoid tumour lymphomatoid granulomatosis, malignant mesothelioma, nasal cavity and paranasal sinus cancer, nasopharyngeal cancer, neuroblastoma, nodal marginal zone B cell lymphoma, non-Hodgkin's lymphoma, oral cavity and oropharyngeal cancer, osteosarcoma, ovarian cancer, pancreatic cancer, penile cancer, pituitary tumours, primary effusion lymphoma, prostate cancer, retinoblastoma, rhabdomyosarcoma, salivary gland cancer, sarcoma, skin cancer (basal and squamous cell, melanoma and merkel cell), small intestine cancer, stomach cancer, testicular cancer, thymus cancer, thyroid cancer, uterine sarcoma, vaginal cancer, vulvar cancer, Waldenstrom macroglobulinemia, and Wilms' tumour. The treatments of the invention are especially effective in the treatment of solid cancers. As such, the subject of the invention may have a solid cancer. The treatments of the invention are particularly effective in the treatment of solid cancers selected from the group consisting of: anal cancer, urinary bladder cancer, breast cancer, cervical cancer, colon cancer, liver cancer, lung cancer (non-small cell and small cell), lung carcinoid tumour, ovarian cancer, pancreatic cancer, penile cancer, prostate cancer, stomach cancer, testicular cancer, uterine sarcoma, vaginal cancer, vulvar cancer, and even more especially for the treatment of breast cancer, colon cancer, liver cancer, lung cancer (non-small cell and small cell), lung carcinoid tumour, pancreatic cancer, prostate cancer, ovarian cancer and urinary bladder cancer.

The treatments of the invention are also especially effective in the treatment of metastatic solid cancers. Metastatic cancer is cancer which has spread from the primary site of origin into one or more different areas of the body.

The cancer may alternatively be any form of hematologic malignancy. A hematologic malignancy is a form of cancer that begin in the cells of blood-forming tissue, such as the bone marrow, or lymphatic system. In many hematologic malignancies, the normal blood cell development process is interrupted by uncontrolled growth of an abnormal type of blood cell. Examples of hematologic cancer include leukaemia, lymphomas, myelomas and myelodysplastic syndromes (lymphomas may be classed as both a solid cancer and a hematologic malignancies). Examples of hematologic malignancies include acute basophilic leukaemia, acute eosinophilic leukaemia, acute erythroid leukaemia, acute lymphoblastic leukaemia, acute megakaryoblastic leukaemia, acute monocytic leukaemia, acute myeloblastic leukaemia with maturation, acute myelogenous leukaemia, acute myeloid dendritic cell leukaemia, acute promyelocytic leukaemia, adult T-cell leukaemia/lymphoma, aggressive NK-cell leukaemia, anaplastic large cell lymphoma, and plasmacytoma, angioimmunoblastic T-cell lymphoma, B-cell chronic lymphocytic leukaemia, B-cell leukaemia, B-cell lymphoma, B-cell prolymphocytic leukaemia, chronic idiopathic myelofibrosis, chronic lymphocytic leukaemia, chronic myelogenous leukaemia, chronic myelomonocytic leukaemia, chronic neutrophilic leukaemia, extramedullary, hairy cell leukaemia, hepatosplenic T-cell lymphoma, Hodgkin's lymphoma, intravascular large B-cell lymphoma, Kahler's disease, lymphomatoid granulomatosis, mast cell leukaemia, multiple myeloma, myelomatosis, nodal marginal zone B cell lymphoma, non-Hodgkin's lymphoma, plasma cell leukaemia, primary effusion lymphoma, and Waldenstrom macroglobulinemia.

Dosing and Dosage Regimens

A therapeutic dose of the phagocytosable particles, or of an injectable composition of the invention, is a dose sufficient to elicit an immune response to a cancer in a subject. For example, a primary immune response and/or a secondary immune response.

In certain embodiments of the invention, the use of phagocytosable particles as described herein for the treatment or prophylaxis of cancer comprises administering a therapeutic dose of phagocytosable particles to a subject. In certain embodiments of the invention, the method of treatment or prophylaxis of cancer of the invention comprises administering a therapeutic dose of phagocytosable particles to a subject. In certain embodiments of the invention, the use of phagocytosable particles of the invention or the method of treatment of the invention comprises administering a therapeutic dose of an injectable composition of the invention to a subject.

The therapeutic dose of phagocytosable particles, or of an injectable composition of the invention, which is required to treat or prevent a cancer in a subject will vary with the route of injection and the characteristics of the subject under treatment, for example the species, age, weight, sex, medical conditions, the particular cancer and its severity, and other relevant medical and physical factors. An ordinarily skilled physician can readily determine and administer the effective amount of phagocytosable particles required for treatment or prophylaxis of a cancer.

A therapeutic dose of phagocytosable particles may be 1 μg to 4000 μg, 10 μg to 3000 μg or 10 μg to 2000 μg. For example, the dose may be 10 μg to 1000 μg, 10 μg to 750 μg, 10 μg to 500 μg, 20 μg to 400 μg, 25 μg to 300 μg, 30 μg to 200 μg, 50 μg to 1000 μg, 50 μg to 750 μg, 50 μg to 500 μg, 50 μg to 400 μg, 50 μg to 300 μg or 50 μg to 200 μg, 100 μg to 1000 μg, 100 μg to 750 μg, 100 μg to 500 μg, 100 μg to 400 μg, 100 μg to 300 μg or 100 μg to 200 μg, 200 μg to 1000 μg, 200 μg to 750 μg, 200 μg to 500 μg, 200 μg to 400 μg, 200 μg to 300 μg, 400 μg to 1000 μg, 400 μg to 750 μg, 400 μg to 500 μg, 500 μg to 1000 μg, 500 μg to 750 μg, or 750 μg to 1000 μg. For example, a dose of phagocytosable particles may be 1, 10, 50, 100, 200, 250, 300, 400, 500, 600, 700, 750, 800, 900, 1000, 1500, 2000, 3000 or 4000 μg.

Preferably, the dose is 100 to 750 μg, more preferably, 200 to 750 μg, more preferably 300 to 750 μg, more preferably 400 to 750 μg, more preferably 500 to 750 μg, more preferably, 600 to 750 μg, even more preferably 650 to 750 μg. For example, a dose of phagocytosable particles may be, 100, 200, 300, 400, 500, 600, 700 or 750 μg.

Alternatively, a therapeutic dose of phagocytosable particles may be determined based on the number of phagocytosable particles. For example, the dose may be approximately 10⁴ to 10¹⁰, 10⁵ to 10⁹, 10⁵ to 10⁸, 10⁵ to 10⁷ or 10⁵ to 10⁶ phagocytosable particles (for example 10⁴, 5⁵, 10⁵, 5⁶, 10⁶, 5⁷, 10⁷, 5⁸, 10⁸, 5⁹, 10⁹, 5¹⁰, or 10¹⁰ phagocytosable particles). Preferably, the dose is approximately 10⁵ to 10⁸, 10⁵ to 10⁷ or 10⁵ to 10⁶ phagocytosable particles, for example approximately 10⁵ to 10⁹, 5×10⁵ to 10⁸, 5×10⁵ to 7.5×10⁷, 5×10⁵ to 5×10⁷, 5×10⁵ to 2.5×10⁷ or 5×10⁵ to 10⁷. More preferably, the dose is approximately 10⁷ to 10⁹, for example approximately 5×10⁷ to 10⁹, 7.5×10⁷ to 10⁹, 7.5×10⁷ to 7.5×10⁸, 7.5×10⁷ to 5×10⁸ or 7.5×10⁷ to 2.5×10⁸ phagocytosable particles. More preferably, the dose is approximately 7.5×10⁷ to 5×10⁸ phagocytosable particles, for example approximately 75 million, 100 million, 150 million, 200 million or 250 million phagocytosable particles.

Alternatively, a therapeutic dose of phagocytosable particles may be determined based on the amount of neoantigenic construct associated to the core. For example, the dose may be 1 μg to 4000 μg, 10 μg to 3000 μg or 10 μg to 2000 μg of neoantigenic construct. For example 1 μg to 4000 μg, 10 μg to 3000 μg or 10 μg to 2000 μg. For example, the dose of neoantigenic construct be 10 μg to 1000 μg, 10 μg to 750 μg, 10 μg to 500 μg, 10 μg to 400 μg, 10 μg to 300 μg, 10 μg to 200 μg, 10 μg to 100 μg or 10 to 50 μg, 10 to 25, 20 μg to 400 μg, 25 μg to 300 μg, 30 μg to 200 μg, 50 μg to 1000 μg, 50 μg to 750 μg, 50 μg to 500 μg, 50 μg to 400 μg, 50 μg to 300 μg or 50 μg to 200 μg, 100 μg to 1000 μg, 100 μg to 750 μg, 100 μg to 500 μg, 100 μg to 400 μg, 100 μg to 300 μg or 100 μg to 200 μg, 200 μg to 1000 μg, 200 μg to 750 μg, 200 μg to 500 μg, 200 μg to 400 μg, 200 μg to 300 μg, 400 μg to 1000 μg, 400 μg to 750 μg, 400 μg to 500 μg, 500 μg to 1000 μg, 500 μg to 750 μg, or 750 μg to 1000 μg. For example, a dose of neoantigenic construct may be 1, 10, 15, 20, 25, 30, 40, 50, 75, 100, 200, 250, 300, 400, 500, 600, 700, 750, 800, 900, 1000, 1500, 2000, 3000 or 4000 μg. Preferably, the dose may be 10 μg to 1000 μg, 10 μg to 750 μg, 10 μg to 500 μg, 10 μg to 400 μg, 10 μg to 300 μg, 10 μg to 200 μg, 10 μg to 100 μg or 10 to 50 μg of neoantigenic construct. Preferably, the dose is 10 to 100 μg of neoantigenic construct, for example 10 μg to 75 μg, 10 μg to 50 μg, 10 μg to 25 μg, 25 μg to 50 μg, or 50 μg to 75 μg of neoantigenic construct. For example, a dose of phagocytosable particles may be, 1, 10, 15, 20, 25, 30, 40, 50, 75, 100 μg of neoantigenic construct. Preferably, the dose is 10 to 50 μg of neoantigenic construct.

A therapeutic dose may be administered as single unit dosage that comprises a therapeutic dose of phagocytosable particles, or as multiple unit dosages when a unit dosage comprise a fraction of a therapeutic dose. Preferably, therapeutic dose of phagocytosable particles of the invention is administered to a subject as a single dose.

A therapeutic dose of phagocytosable particles, or of an injectable composition comprising a therapeutic dose of phagocytosable particles of the invention, may be administered to a subject once.

In certain preferred embodiments, a subject is administered a therapeutic dose of phagocytosable particles, or of an injectable composition comprising a therapeutic dose of phagocytosable particles, and is then administered at least one further (or “subsequent”) therapeutic dose of phagocytosable particles of the invention, or an injectable composition comprising a therapeutic dose of phagocytosable particles of the invention. Further (or “subsequent”) therapeutic doses of phagocytosable particles may be administered daily, every second or third day, weekly, every second, third or fourth week, monthly, every second, third or fourth month, every 6 months, or every year. The number and frequency of further therapeutic dose(s) of phagocytosable particles of the invention, or an injectable composition comprising a therapeutic dose of phagocytosable particles of the invention, will depend on the subject, and the form and severity of the cancer to be treated.

In one embodiment, the use of phagocytosable particles for the treatment or prophylaxis of cancer comprises administering one or more subsequent therapeutic doses of phagocytosable particles, or of an injectable composition comprising a therapeutic dose of phagocytosable particles of the invention, to the subject, wherein the subject is one whom has previously been administered a therapeutic dose of the phagocytosable particles, or an injectable composition comprising a therapeutic dose of phagocytosable particles of the invention. Each of the one or more subsequent therapeutic doses are a dose sufficient to elicit an immune response towards a cancer cell in the subject (i.e. a primary immune response and/or a secondary immune response).

In embodiments of the invention comprising administering one or more subsequent therapeutic doses of phagocytosable particles or of an injectable composition comprising a therapeutic dose of phagocytosable particles of the invention, to the subject, preferably 2, 3, 4, 5, 6, 7, 8, 9, 10 or n subsequent therapeutic doses of the phagocytosable particle or injectable composition are administered to the subject; wherein “n” is any number of doses greater than 10 doses (for example 11, 12, 13, 14, 15, 16, 17, 18, 19, 20, 21, 22, 23, 24, 25, 26, 27, 28, 29 or 30 doses). Preferably, the number and frequency of subsequent therapeutic doses administered to the subject is sufficient to treat or prevent cancer in the subject.

A therapeutic dose of phagocytosable particles, or of an injectable composition of the invention, may independently have any of the properties and/or characteristics of a therapeutic dose of phagocytosable particles, or of an injectable composition, as described herein. In preferred embodiments of the invention, the phagocytosable particles administered to the subject as a subsequent therapeutic dose comprises the same type(s) of neoantigenic construct as the phagocytosable particles previously administered to the subject as a therapeutic dose. The core of the phagocytosable particle administered as a subsequent therapeutic dose may be the same or different to the core of the phagocytosable particles previously administered to the subject as a therapeutic dose (e.g. the cores may have different sizes and/or comprise different materials/polymers as described herein). Preferably, the phagocytosable particles administered as a subsequent therapeutic dose may comprise the same type(s) of neoantigenic construct and the same core as the phagocytosable particles previously administered to the subject as a therapeutic dose (i.e. the phagocytosable particles are of the same set, and are the same as each group(s) as those previously administered to the subject as a therapeutic dose).

In embodiments of the invention comprising administering one or more subsequent therapeutic doses of phagocytosable particles or of an injectable composition comprising a therapeutic dose of phagocytosable particles, to the subject, preferably, the one or more subsequent therapeutic doses are administered to the subject at intervals of days, weeks or months. For example, one or more subsequent therapeutic doses are administered to the subject every day, every second day, every third day, every fourth day, every fifth day, every sixth day. Alternatively, or additionally, one or more subsequent therapeutic doses are administered to the subject, for example, once every week, once every two weeks, once every three weeks or once every four weeks. Alternatively, or additionally, one or more subsequent therapeutic doses are administered to the subject, for example, once every month, once every two months, once every three months or once every 6^{th} month. Alternatively, or additionally, one or more subsequent therapeutic doses are administered to the subject, for example, once every year.

In one embodiment of the invention, the one or more subsequent therapeutic doses are administered to the subject once every year, two times every year or three times every year. For example, the one or more subsequent therapeutic doses may be administered to the subject once every year, two times every year or three times every year fora period of 1 to 10 years, 1 to 20 years, 1 to 30 years, 1 to 40 years or 1 to 60 years (for example for a period of 1 to 10 years, 10 to 20 years, 20 to 30 years, 30 to 40 years or 50 to 60 years).

The present inventors have found that by administering one or more subsequent therapeutic doses to a subject over a long period of time (i.e. over one or more years), it is possible to boost the number of anticancer memory B-cells and memory T-cells that are present in the subject. It is expected that by boosting the number of anticancer memory B-cells and memory T cells in a subject, the anticancer immunological memory in the subject is maintained, such that a cancer is prevented from growing (for example, forming a tumour) or returning.

In certain embodiments of the invention, a booster dose is administered to a subject whose cancer has been successfully treated (with one or more therapeutic doses as described herein) to prevent the cancer returning.

A booster dose of phagocytosable particles, or of an injectable composition of the invention, may independently have any of the properties and/or characteristics of a therapeutic dose of phagocytosable particles, or of an injectable composition, as described herein. In preferred embodiments of the invention, the phagocytosable particles administered to the subject as a booster dose comprise the same type(s) of neoantigenic construct as the phagocytosable particles administered to the subject as a therapeutic dose. The core of the phagocytosable particle may be the same or different to the core of a phagocytosable particle administered to the subject as a therapeutic dose (e.g. the cores may have different sizes and/or comprise different materials/polymers as described herein). Preferably, a booster dose comprises phagocytosable particles having the same type(s) of neoantigenic construct and the same core as the phagocytosable particles administered to the subject as a therapeutic dose (i.e. the phagocytosable particles are of the same set, and are the same as each group(s) as those previously administered to the subject as a therapeutic dose).

In one embodiment of the invention, one or more booster doses are administered to the subject, for example, once every week, once every two weeks, once every three weeks or once every four weeks. Alternatively, or additionally, one or more booster doses are administered to the subject, for example, once every month, once every two months, once every three months or once every 6^{th} month. Alternatively, or additionally, one or more booster doses are administered to the subject, for example, once every year.

In another embodiment of the invention, the one or more booster doses are administered to the subject once every year, two times every year or three times every year. For example, the one or more booster doses may be administered to the subject once every year, two times every year or three times every year for a period of 1 to 10 years, 10 to 20 years, 20 to 30 years, 30 to 40 years or 50 to 60 years.

It is expected that boosting the number of anticancer memory B-cells and memory T cells in a subject by administering one or more booster doses maintains the anticancer immunological memory in the subject, such that the cancer is prevented from growing or returning.

Ex-Vivo Activation and Expansion of Anticancer T-Cells

The present invention also provides a treatment of the invention which also comprises the additional steps of: a) harvesting APCs and anticancer T-cells from the subject; b) expanding the anticancer T-cells harvested from the subject; and c) administering a therapeutic dose of the expanded anticancer T-cells to the subject.

The present invention also provides a method of treating or preventing cancer in a subject which comprises the additional steps of: a) harvesting APCs and anticancer T-cells from the subject; b) expanding the anticancer T-cells harvested from the subject; and c) administering a therapeutic dose of the expanded anticancer T-cells to the subject.

Step a): Harvesting APCs and Anticancer T-Cells from the Subject after the Administration of the Phagocytosable Particle to the Subject:

In one embodiment of the invention, in step a) APCs and anticancer T-cells are harvested from a subject following administration of a dose (preferably a therapeutic dose) of phagocytosable particles or of an injectable composition of the invention to the subject. Alternatively, or additionally, APCs and anticancer T-cells may be harvested from a subject at the same time and/or before administrating a dose (preferably a therapeutic dose) of phagocytosable particles or of an injectable composition of the invention to the subject. Preferably, APCs and anticancer T-cells are harvested from a subject after administering a dose (preferably a therapeutic dose) of phagocytosable particles or of an injectable composition of the invention to the subject.

The APCs must be compatible with the anticancer T-cells, such that they are capable of presenting antigens to the anticancer T-cells in an antigen-specific context (MHC restricted) that the anticancer T-cells can react to. The APCs and anticancer T-cells are preferably obtained from the same species and donor-matched with respect to MHC receptors. However, use of genetically engineered APCs from a different species is also envisioned. More preferably the APCs and anticancer T-cells are obtained from the same subject. If the APCs and anticancer T-cells are derived from the same subject, any potential for a mismatch between the APCs and anticancer T-cells is avoided.

The APCs harvested from a subject may comprise phagocytes, monocytes and/or dendritic cells. The anticancer T-cells may comprise CD4+ and/or CD8+ T-cells.

The APCs and anticancer T-cells may be harvested from a blood sample derived from a subject. Preferably the blood sample is a peripheral blood mononuclear cell (PBMC) sample. PBMCs are a fraction of human blood prepared by density gradient centrifugation of whole blood. The PBMCs mainly consists of lymphocytes (70-90%) and monocytes (10-30%), while red blood cells, granulocytes and plasma have been removed. Monocytes may in some instances make up 10 to 20% of the cell numbers in a PBMC sample, for example 10 to 15%.

APCs and anticancer T-cells may also be derived from a tumour of the subject, for example a sample derived from a lymphatic vessel in a tumour or a tumour draining lymph node (i.e. a sentinel node). Preferably, the APCs and anticancer T-cells are harvested from the same sample derived from the subject and/or the same tumour of the subject. Alternatively, or additionally, APCs and anticancer T-cells may be harvested from different samples derived from the subject. For example, the APCs may be harvested from a blood sample and the anticancer T-cells may be harvested from a tumour.

Preferably, the APCs and anticancer T-cells are harvested from a PBMC sample. For example, the APCs and anticancer T-cells are harvested from the same PBMC sample or from different PBMC samples. Obtaining PBMCs from peripheral blood samples is a routine protocol, which provides a convenient source for both APCs and T-cells at the same time and from the same subject. The PBMC sample may be freshly used or it may be subjected to freezing for storage before use.

Step b): Expanding the Anticancer T-Cells Harvested from the Subject:

The APCs and anticancer T-cells harvested from the subject in step a) may be used in the preparation of anticancer T-cells suitable for use in the treatment or prophylaxis of cancer in the subject. The anticancer T-cells that are suitable for administration to the subject are prepared by in vitro activation and expansion of anticancer T cells harvested from the subject. The in vitro activation and expansion method may comprise the steps of:
i) providing a phagocytosable particle phagocytosable particle comprising a core and a neoantigenic construct tightly associated to the core, wherein the neoantigenic construct comprises a neoepitope peptide having an amino acid sequence corresponding to an amino acid sequence of a part of a protein or peptide known or suspected to be expressed by a cancer cell in the subject, wherein the part of the protein or peptide has at least one somatic mutated amino acid;
ii) providing an APC;
iii) contacting the phagocytosable particle with the APC in vitro and under conditions allowing phagocytosis of the phagocytosable particle by the APC;
iv) providing anticancer T-cells harvested from the subject;
v) contacting the anticancer T-cells with the APC from step iii) in vitro, and under conditions allowing specific activation and expansion of anticancer T-cells in response to neoepitopes presented by the APC.

The in vitro expansion method may comprise contacting APCs with 100 to 1×10⁹ phagocytosable particles, for example 100 to 1×10⁸, for example 100 to 1×10⁷ phagocytosable particles are used in a given expansion run, for example 1000 to 1×10⁷ phagocytosable particles. For example, the ratio of phagocytosable particle to APC is in the range 1000:1 to 1:10. The ratio can be optimised depending on the size of the phagocytosable particle. For example, for a phagocytosable particle with a largest diameter of about 1 μm, the ratio may be in the range 50:1 to 2:1, for example 25:1 to 5:1, 15:1 to 7:1, and 10:1.

For the avoidance of doubt, phagocytosable particles suitable for contacting an APC, may independently have any of the properties and/or characteristics of the phagocytosable particles administered as a therapeutic dose or booster dose to a subject.

In certain embodiments of the invention, an APC is contacted with a phagocytosable particle(s) that comprises the same type(s) of neoantigenic construct as the phagocytosable particles administered to the subject as a therapeutic dose. The core of the phagocytosable particle may the same or different to the core of a phagocytosable particle administered to the subject as a therapeutic dose (e.g. the cores may have different sizes and/or comprise different materials/polymers as described herein). In preferred embodiments of the invention, an APC is contacted with a phagocytosable particle(s) that comprises the same type(s) of neoantigenic construct and the same core as the phagocytosable particles administered to the subject in the therapeutic dose.

In one embodiment of the invention, the method of expanding anticancer T-cells comprises adding low doses IL-2 to the anticancer T-cell sample, for example greater than 1.25 U/ml (for example 1.25 U/ml, 2.5 U/ml, 5 U/ml, or 50 U/ml), preferably greater than 2.5 U/ml, 5 U/ml, or 50 U/ml. Antigen specific T-cell expansion occurs in the presence of the antigen-presenting cell when IL-2 is simultaneously present. The IL-2 promotes the differentiation of anticancer T-cells into effector anticancer T-cells and into memory anticancer T-cells. Following expansion of anticancer T-cells, the APCs may be removed from the expanded T-cell population, for example by magnetic separation.

In another embodiment of the invention, the method of expanding anticancer T-cells comprises adding IL-2 and/or IL-7 and/or IL-15 to the anticancer T-cell sample, for example a low dose of IL-2 to the anticancer T-cell sample, for example greater than 1.25 U/ml (for example 1.25 U/ml, 2.5 U/ml, 5 U/ml, or 50 U/ml), preferably greater than 2.5 U/ml, 5 U/ml, or 50 U/ml of IL-2, with optional addition of IL-7 and/or IL-15. For example a low dose of IL-7 to the anticancer T-cell sample, for example greater than 1.25 U/ml (for example 1.25 U/ml, 2.5 U/ml, 5 U/ml, or 50 U/ml), preferably greater than 2.5 U/ml, 5 U/ml, or 50 U/ml of IL-7; and/or for example, a low dose of IL-15 to the anticancer T-cell sample, for example greater than 1.25 U/ml (for example 1.25 U/ml, 2.5 U/ml, 5 U/ml, or 50 U/ml), preferably greater than 2.5 U/ml, 5 U/ml, or 50 U/ml of IL-15.

In preferred embodiments of the invention, the method of activating and expanding anticancer T-cells in vitro comprises a step of removing the APCs that have internalised a phagocytosable particle of the invention from the anticancer T-cells. In embodiments of the invention, wherein the phagocytosable particles of the invention comprises a magnetic core, the APCs are removed from the anticancer T-cells by using magnetic separation.

Following contact of an anticancer T-cell with an APC that has internalised a phagocytosable particle of the invention, the degree of anticancer T-cell activation may be determined, for example by comparing the degree of anticancer T-cell activation to a relevant reference. Determining the degree of anticancer T-cell activation may be performed using T-cell activation assays known in the art, for example an ELISpot, FluoroSpot, intracellular staining of cytokines with flow cytometry, FASCIA (Flow-cytometric Assay for Specific Cell-mediated Immune-response in Activated whole blood), proliferation assays (e.g. thymidine incorporation, CFSE or BrdU staining), specific TCR-detection with MHC-I or II tetramers, and ELISA- or Luminex analysis of secreted cytokines ELIS potassays. The method may comprise the step of comparing the degree of anticancer T-cell activation to a relevant reference. Suitable references include, for example, a T-cell sample that does not comprising anticancer T-cells, or an anticancer T-cell sample that has not been contacted with an APC that has internalised a phagocytosable particle.

c) Administering a Therapeutic Dose of the Expanded Anticancer T-Cells to the Subject.

A therapeutic dose of the expanded anticancer T-cells may be administered to the subject. Thus, the present invention also provides anticancer T-cells for use in treating or preventing cancer in a subject. The expanded anticancer T-cells may be administered to the subject intravenously, intraarterially, intrathecally or intraperitoneally.

The precise dosage of the expanded anticancer T-cells will vary with the dosing schedule, the age, size, sex and condition of the subject (typically mammal or human), the nature and severity of the condition, and other relevant medical and physical factors. Thus, a precise therapeutically effective amount can be readily determined by the clinician. An appropriate amount can be determined by routine experimentation from animal models and human clinical studies. For humans, an effective dose will be known or otherwise able to be determined by one of ordinary skill in the art.

### EXAMPLES

### Example 1: General Protocol for Coupling Neoantigenic Constructs or Model Peptides/Proteins to Magnetic Cores

Coupling of Neoantigenic Constructs or Model Peptides/Proteins to a Core:

Dynabeads® MyOne™ Carboxylic Acid (ThermoFischer Scientific) were used (1 μm diameter spheres) as the core. Dynabeads® MyOne™ Carboxylic Acid particles are paramagnetic polystyrene particles comprising iron oxide and functionalised on the surface of the particle with free carboxylic acid groups. The coupling procedure was carried out according to the manufacturer's protocol (Two-Step procedure using NHS (N-Hydroxysuccinimide) and EDC (ethyl carbodiimide)):

Step 1): The polystyrene particles are washed twice with MES-Buffer (25 mM MES (2-(N-morpholino)ethanesulfonic acid), pH 6). The carboxylic acid groups were then activated by adding 50 mg/ml NHS (N-Hydroxysuccinimide) and 50 mg/ml EDC (N-(3-Dimethylaminopropyl)-N′-ethylcarbodiimide) in MES-buffer to the polystyrene particles and incubated for 30 min at room temperature (RT). The polystyrene particles were collected with a magnet and the supernatant was removed and the polystyrene particles washed twice with MES-buffer.

Step 2): The neoantigenic construct or model peptide/protein sample was diluted in MES-buffer to a concentration of 1 mg/ml, total 100 μg and added to the polystyrene particles and incubated for 1 h at RT. The polystyrene particles were collected with a magnet and the supernatant was removed and saved for peptide-concentration measurement. The non-reacted activated carboxylic acid groups were quenched with 50 mM Tris pH 7.4 for 15 min. The polystyrene particles were then washed with PBS pH 7.4 and then stored in −80° C.

A BCA (bicinchoninic acid) protein assay kit (Pierce BCA Protein Assay Kit, ThermoFisher Scientific) was used according to the manufacturer's protocol, to measure the amount of peptidic material coupled to the polystyrene particles and to measure the peptidic material concentration of the neoantigenic construct sample before coupling as well as the peptidic material concentration of the supernatant after coupling.

Several polypeptides were tested and an estimated average of 48.7 μg (mean: 48.7, SD: 20.5, N=10) neoantigenic construct was coupled per 1 mg polystyrene particles. According to manufacturer's instruction, 50 μg polypeptide can be coupled per 1 mg particles, indicating that the efficiency of the coupling achieved was high.

### Example 2: Washes

Polystyrene particles were coupled to recombinant neoantigenic constructs produced in E. coli according to the method described in Example 1. After coupling, the polystyrene particles were washed with one of 3 different wash-buffers: 2M NaOH pH 14.3, 8 M Urea or 6 M Guanidine (Guanidine-HCl), all in sterile water at RT, or they were incubated in PBS at 95° C. The polystyrene particles were suspended in the buffer and shaken for 4 min, collected with a magnet and the supernatant removed. This was repeated 3 times. The heat treated polystyrene particles were put in PBS pH 7.4 and put in a heating block at 95° C. for 5 minutes, then collected with a magnet and the supernatant removed. This was repeated 3 times. The particles were then washed 3 times with sterile PBS to remove any remaining wash-buffer.

Four different washing conditions were tested: (a) High pH (2M NaOH pH 14.3), (b) Heat (95° C.) and sterilising/denaturing agents ((c) 8M Urea and (d) 6M guanidine hydrochloride). In every case, the neoantigenic constructs associated with the polystyrene particles remained associated with the polystyrene particles.

### Example 3a: Identification of Suitable Particle Size for Phagocytosable Particles

A cell proliferation assay measuring Thymidine incorporation was used to test the effect of phagocytosable particle size on antigen-specific T-cell activation. Splenocytes from ovalbumin (OVA) immunized mice were stimulated with OVA coupled polystyrene particles of different sizes to measure antigen specific proliferation.

Dynabeads® MyOne™ Carboxylic Acid particles with a diameter of 5.6 μm, 1 μm and 0.2 μm were coupled with OVA (OVA-particles) or bovine serum albumin (BSA-particles) according to the protocol in Example 1.

To test the effectiveness of the OVA-particles to stimulate antigen specific T-cell activation a proliferation assay (with ³H thymidine incorporation) was used. Particle concentration in relation to cell concentration was 1:1 for the 5.6 μm particles, 10:1 for the 1 μm particles and 500:1 for the 0.2 μm particles. Total protein concentration during the incubation with the cells was calculated to 125 ng/ml, 160 ng/ml and 160 ng/ml for the 5.6 μm, 1 μm and 0.2 μm respectively. The proliferation assay was run as follows:

As stimuli, ovalbumin (SigmaAldrich) and BSA (SigmaAldrich) coupled to Dynabeads® MyOne™ Carboxylic Acid particles were used (OVA-particles or BSA-particles). Mice were immunized to ovalbumin via monthly injections of 100 μg ovalbumin (Sigma) adsorbed to aluminium hydroxide. Three months after the first injection the mice were killed and spleens harvested. Splenocytes were prepared by standard procedures, as described in Thunberg et al. 2009, Allergy 64:919.

The cells were incubated in cRPMI either with OVA-particles or BSA-particles (10 particles per cell) for 5 days. All cells were incubated for 6 days in a humidified atmosphere with 6% CO₂ at 37° C. One μCu/well [³H] thymidine was added to cell cultures for the final 18 h of incubation. Mean counts per minute (cpm) obtained from stimulated triplicates were divided by mean cpm values from un-stimulated cells and expressed as stimulation indices (SI). SI-values ≥2.0 are generally considered positive.

As seen in FIG. 1, cells incubated with OVA-particles with a diameter of 0.2 μm showed increase in proliferation with a mean SI of 4.1 (95% Cl 2.4-5.8, P=0.007). The cells incubated with OVA-particles with a diameter of 1 μm showed increase in proliferation with a mean SI of 8.4 (95% Cl 6.1-10.6, P<0.005). The cells incubated with OVA-particles with a diameter of 5.6 μm failed to stimulate proliferation, mean Sl 1.1 (95% Cl 0.4-2.7, P=0.876).

These results show that antigen coupled to particles of different sizes can stimulate cell proliferation. The particles with a diameter of about 1 μm seems to be most efficient in regards to cell stimulation but particles down to a size of 0.2 μm still works. It is reasonable to predict that particles of sizes larger than 1 μm also work, although as the diameter comes close to 5.6 μm the particles completely fail to stimulate the cells. It is reasonable to assume that 1 μm is an optimal size, since it is similar to the size of bacteria. Our immune system has evolved to phagocytose and react to microorganisms/particles of this size. A normal antigen presenting cell has a size in the range 10-15 μm.

### Example 3b: Comparison of Antigen Coupled Particles of Different Particle Sizes and their Effectiveness in Activating and Expanding T-Cells

(i) Preparation of Antigen Coupled Phagocytosable Particles:

Three kinds of paramagnetic polystyrene phagocytosable particles of different sizes were used:
diameter of 1 μm (Dynabeads MyOne Carboxylic Acid, ThermoFisher),
diameter of 2.8 μm (Dynabeads M-270 Carboxylic acid, ThermoFisher) and
diameter of 4.5 μm (Dynabeads M-450 Epoxy, ThermoFisher).

The phagocytosable particles were coupled with the model antigen Cytomegalovirus (CMV) protein pp65 construct (SEQ ID NO: 19) according to the manufacturer's instruction. To remove endotoxin, the phagocytosable particles were washed five times with a 0.75M sodium hydroxide buffer and subsequently resuspended in sterile PBS.

(ii) Incubation of Antigen Coupled Phagocytosable Particles:

Peripheral blood mononuclear cells (PBMCs) from a CMV-sensitive healthy donor, isolated via standard ficoll-based density gradient centrifugation were cultured together with the phagocytosable particles coupled with the CMV construct (hereinafter referred to as “CMV-particles”) in a 48-well plate for 18 h at 37° C., 5% CO₂, 500,000 cells/well at a concentration of 1,000,000 cells/ml. The concentration of CMV-particles were equalized based on total surface area (a surrogate marker for CMV amount as it is bound to the surface of the CMV-particles). This equalled to 10 CMV-particles/PBMC for the 1 μm sized particles, 1.4 CMV-particle/PBMC for the 2.8 μm sized particles and 0.5 CMV-particles/PBMC for the 4.5 μm sized particles, based on the number of total PMBCs in the sample. The results for each particle sized is shown below in Table 1.

TABLE 1

| | Number of | Ratio of CMV- | Number of CMV- |
|---|---|---|---|
| Particle | PBMCs in | particles | particles |
| size | sample | to PBMCs | in sample |
| 1 μm | 500,000 | 10:1 | 5,000,000 |
| 2.8 μm | 500,000 | 1.4:1 | 700,000 |
| 4.5 μm | 500,000 | 0.5:1 | 250,000 |

(iii) Assessment of Uptake:

After incubation, the number of phagocytosed CMV-particles were manually counted using a confocal microscope. Eight cells were counted to obtain mean and standard deviation values. In FIG. 5A, there are shown images from the confocal microscope of representative cells with intracellular phagocytosed CMV-particles. Black dashed line indicates the outline of the cell. The white line shows the dimensions of the total intracellular CMV-particles.

This method was not applicable for the 1 μm CMV-particles as they were too small to accurately count. In order to assess the amount of 1 μm CMV-particles, the total volume of all phagocytosed CMV-particles were measured and the amount of individual CMV-particles were back-calculated based on the total volume, assuming a packing density of 60%. This method proved reasonably accurate for the 2.8 μm CMV-particles (manually counted 9.1 CMV-particles/cell vs estimated 11.9 CMV-particles/cell) and 4.5 μm CMV-particles (manually counted 3.1 CMV-particles/cell vs estimated 2.4 CMV-particles/cell) and can as such be assumed to accurately estimate the amount of 1 μm CMV-particles as well.

The uptake of CMV-particles in shown in FIGS. 5B and 5C. FIG. 5B shows the number of CMV-particles taken up by each cell as assessed by manual counting (8 cells counted per bead-type). Using the manual counting method, it was found that the number of phagocytosed particles per cell for the 4.5 μm CMV-particles was of 3.1 (±1.1). For the 2.8 μm CMV-particles it was 9.1 (±2.2). It was not possible to count the number of 1 μm CMV-particles using this method.

FIG. 5C shows the number of CMV-particles taken up by each cell as assessed by the volume calculation (3 cells measured per bead-type) (*p<0.05 **p<0.01 ***p<0.001, calculated using Students T-test). Using the volume calculation method, it was found that the number of phagocytosed particles per cell for the 4.5 μm CMV-particles was of 2.4 (±1.1). For the 2.8 μm CMV-particles it was 11.9 (±3.2). For the 1 μm CMV-particles it was 203.7 (±21.9).

Based on the number of CMV-particles taken up by each cell as assessed by the volume calculation method, the total phagocytized surface area, and by extension the total amount of CMV, was calculated. The surface area that was taken up was calculated as 639.6 (±68.9) μm² for the 1 μm CMV-particles, 293.1 (±79.3) μm² for the 2.8 μm CMV-particles and 150.7 (±67.0) μm² for the 4.5 μm CMV-particles. These data are shown in Table 2 below.

TABLE 2

| | | CMV-particles | |
|---|---|---|---|
| | CMV-particles | uptake per cell | Surface area |
| Particle | uptake per cell | (calculated | taken up |
| size | (counted) | from volume) | per cell |
| 1 μm | — | 203.7 (±21.9) | 639.6 (±68.9) μm² |
| 2.8 μm | 9.1 (±2.2) | 11.9 (±3.2) | 293.1 (±79.3) μm² |
| 4.5 μm | 3.1 (±1.1) | 2.4 (±1.1) | 150.7 (±67.0) μm² |

(iv) Assessment of T-Cell Stimulation

The ability of the antigen coupled particles to stimulate T-cells and hence promote their expansion was assessed by measuring the release of IFNγ, IL22 and IL17A from PBMCs using a FluoroSpot assay (Mabtech, Sweden). PBMCs (250,000/well) from CMV-sensitive healthy donors (n=2) were stimulated with the CMV-particles in triplicates. The concentration of antigen coupled particles were as previously described equalized based on total surface area: 10×1 μm CMV-particles/cell, 1.4×2.8 μm CMV-particles/PBMC and 0.5×4.5 μm CMV-particles/cell. The number of PBMCs per well of the FluoroSpot assay is shown below (Table 3) for each particle size, together with the estimate number of monocytes per well (based on an estimated 20% monocyte content of a PBMC sample).

TABLE 3

| | Number of | Number of |
|---|---|---|
| particle | PBMCs | monocytes |
| size | per well | per well |
| 1 μm | 250,000 | 50,000 |
| 2.8 μm | 250,000 | 50,000 |
| 4.5 μm | 250,000 | 50,000 |

The PBMCs were incubated for 44 h at 37° C., 5% CO₂. The plates were developed according to the manufacturer's instructions and read in an automated FluoroSpot reader. The data reported for the FluoroSpot is spot-numbers when the cells are stimulated with CMV-particles above the spot-numbers when not stimulated with CMV-particles.

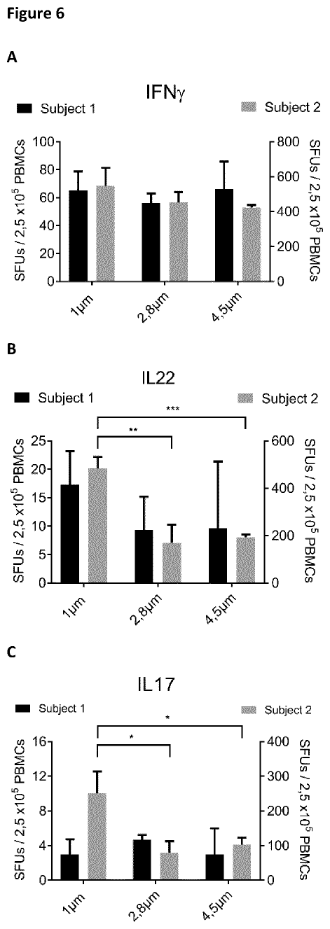

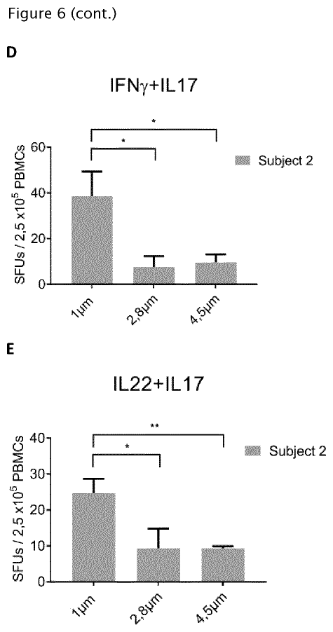

The level of IFNγ-production, as assessed in the FluoroSpot assay, is shown in FIG. 6A. It is seen that there is little difference between the CMV-particles.

The level of IL22 and IL17 production, as assessed in the FluoroSpot assay, is shown in FIGS. 6B and 6C. It is seen that the 1 μm CMV-particles caused a significantly higher IL22 and IL17 production in one individual than the larger CMV-particles, with a similar trend seen for the other individual in regards to IL22.

The level of dual-cytokine production, as assessed in the FluoroSpot assay, is shown in FIGS. 6D and 6E. It is seen that the 1 μm CMV-particles caused a significantly higher dual-cytokine release (IFNγ+IL17 and IL22γ+IL17) for one healthy donor when stimulated with the 1 μm antigen coupled particles than with the larger CMV-particles.

The cytokine release in these experiments serves as a proxy for T-cell expansion. In general, IFNγ is produced by CD4+ T-cells (Th1 subclass) and CD8+ T-cells. IL17 and IL22 are mainly produced by pro-inflammatory Th17 CD4+ T-cells. Such cells are pro-inflammatory have been shown to assist in tumour eradication. The data suggest that the 1 μm beads activate and cause expansion of Th1 CD4+ T-cells and CD8+ T-cells to the same degree as the other beads, with the added benefit of also activating and causing expansion of additional pro-inflammatory Th17 CD4+ T-cells and the less distinct but still pro-inflammatory double cytokine producing T-cells.

### Example 4: Prophylactic Effect of Phagocytosable Particles in a Mouse Cancer Model

Two groups of phagocytosable particles were used in this experiment: 1) phagocytosable particles comprising a polystyrene particle core and comprising neoantigenic construct M272120 (SEQ ID NO: 1) tightly associated to the core; and 2) phagocytosable particles comprising a polystyrene particle core and neoantigenic construct M304748 (SEQ ID NO: 2) tightly associated to the core. The phagocytosable particles were prepared using the method described herein in Example 1 and 2: the neoantigenic constructs were coupled to 1 μm superparamagnetic beads (Sera-Mag SpeedBeads (hydrophilic) Carboxylate-Modified Magnetic particles, GE Healthcare) following the protocol outlined in Examples 1 and 2. The neoantigenic construct design was based on previously published studies using the B16-F10 tumour model (Kreiter et al. (2015), Nature 520:692 and Castle et al. (2012) Cancer Res 72:1081). Once made, the two groups of phagocytosable particles were mixed (1:1 ratio), then purified and a stock solution of the particles in PBS with CpG oligodeoxynucleotide (ODN 1668, Enzo) as adjuvant was made. The stock solution had a concentration of 10 million particles/μl.

Healthy mice (C57BL/6 mice) were administered with either a low dose (1 μl of phagocytosable particles in 9 μl of PBS) or high dose (10 μl of phagocytosable particles) of phagocytosable particles by injection into either an inguinal lymph node or subcutaneously. Each dose and route of administration was assessed in triplicate (n=3). The stock phagocytosable particle sample used to prepare the low and high doses contained 10 million beads/μl. Therefore, the low dose contained approximately 10 million phagocytosable particles, and the high dose contained approximately 100 million phagocytosable particles. Each dose of phagocytosable particles contained two different groups of phagocytosable particles: 1) phagocytosable particles comprising a polystyrene particle core and comprising neoantigenic construct M272120 (SEQ ID NO: 1) tightly associated to the core; and 2) phagocytosable particles comprising a polystyrene particle core and neoantigenic construct M304748 (SEQ ID NO: 2) tightly associated to the core.

Mice were administered a first dose of the phagocytosable particles on the first day, and a second dose was then administered to the same mice approximately 1 month later (33 days later). Blood samples were harvested from the mice around 3 weeks after administration of first dose (22 days) and around 3 weeks after the second dose of phagocytosable particles (23 days).

The wellbeing of the mice that received two doses of phagocytosable particles via inguinal lymph node injection was also assessed. The inventors found that 2×10 ul injections of phagocytosable particles via the inguinal lymph node did not affect the animal wellbeing (body weight and overall health status). The inguinal lymph node and mice spleens also showed no macroscopic abnormalities, suggesting that the phagocytosable particles were well tolerated by the mice.

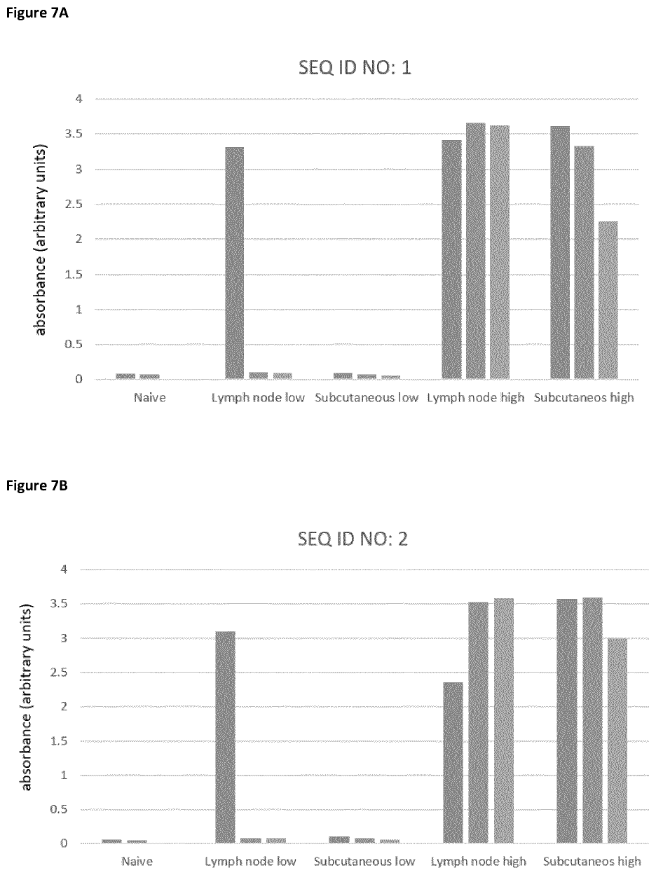

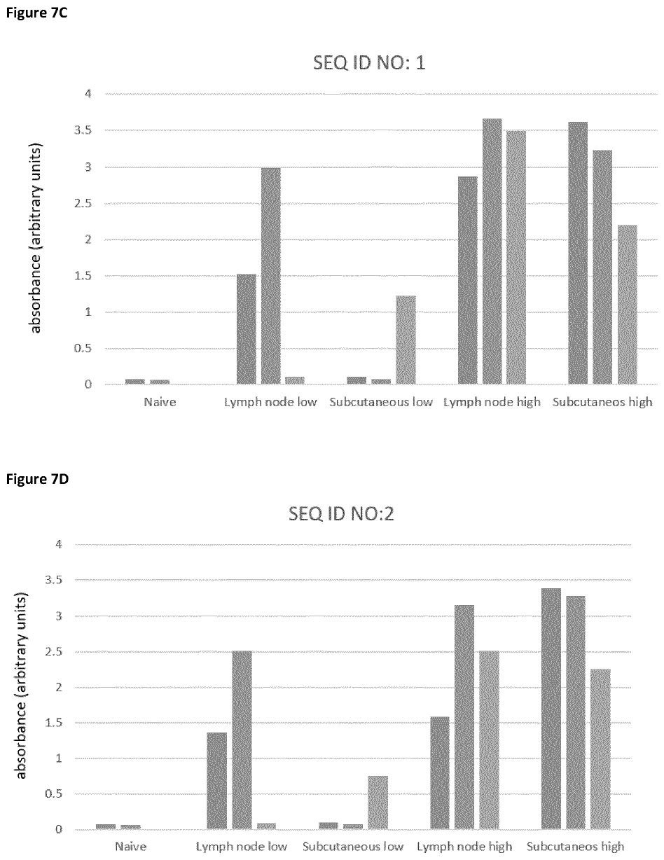

The harvested blood samples were analysed using an enzyme-linked immunosorbent assay (ELISA). The assay was performed in 96 well plates. The plates were coated with neoantigenic constructs M272120 (SEQ ID NO: 1) or M304748 (SEQ ID NO: 2) by addition of 100 ul of the neoantigenic construct at a concentration of 5 μg/ml in PBS. The plates were incubated overnight before washing. The coated 96 well plates were then incubated with 100 ul of diluted (1:1000) mouse blood serum harvested from the mice following administration of either the first dose or second dose of the phagocytosable particles. The plates were then washed, followed by incubated with an anti-mouse IgG-HRP secondary antibody (Jackson Labs, diluted 1:8000). Finally, the plates were washed and then developed with TMB substrate solution (Sigma-Aldrich) and the absorbance measured at 370 and 650 nm. The absorbance for each mouse blood serum sample for each neoantigenic construct is shown in FIG. 7A (blood serum harvested from mice following the first dose; neoantigenic construct=M272120 (SEQ ID NO: 1)), Figure B (blood serum harvested from mice following the first dose; neoantigenic construct=M304748 (SEQ ID NO: 2)), FIG. 7C (blood serum harvested from mice following the second dose; neoantigenic construct=M272120 (SEQ ID NO: 1)), and FIG. 7D (blood serum harvested from mice following the second dose; neoantigenic construct=M304748 (SEQ ID NO: 2)). As a control sample, blood serum samples were taken from naïve mice (n=3, no dose of phagocytosable particles administered) were also analysed. As shown by FIGS. 7A, 7B, 7C and 7D, mice having received a high dose of the phagocytosable particles (administered into lymph node or subcutaneously) had a greater proportion of anti-neoepitope antibodies in the blood serum samples compared to the blood serum samples harvested from the mice that received a low dose of phagocytosable particles via the same route and the mice that did not receive a dose of phagocytosable particles (i.e. naïve mice).

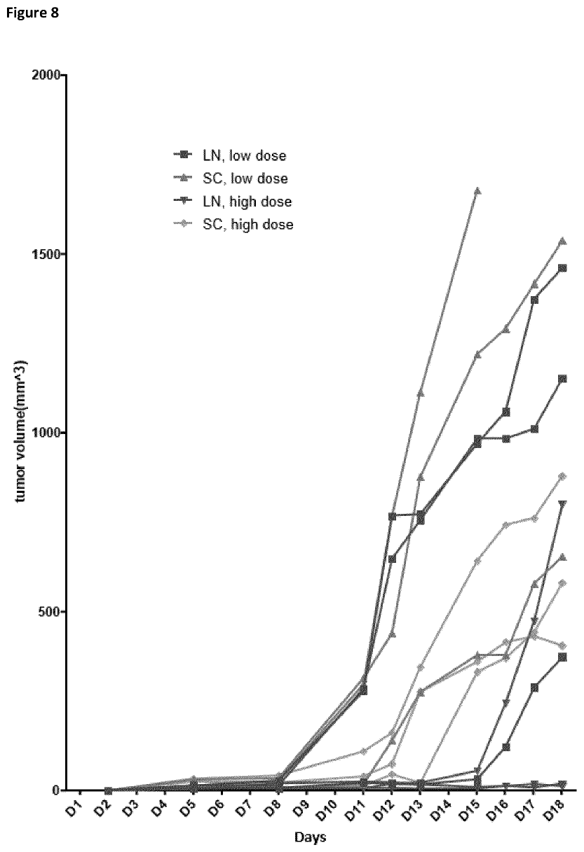

Approximately 2 months after administration of the second dose of phagocytosable particles (54 days), the mice were subcutaneously injected with 500,000 cells of the melanoma cancer cell line B16-F10. Tumour volume was measured daily. FIG. 8 shows the increase in tumour volume over time (days, D).

### Example 5: A Pilot Study Utilising the Phagocytosable Particles for Ex-Vivo Expansion of Anticancer T-Cells

(i) Identification of Neoepitope Targets in Urinary Bladder Cancer

Urinary bladder cancers display a high rate of mutations, thus expressing a large number of neoepitopes that could potentially be recognized as non-self by the immune system. As such, the inventors investigated tumour polymorphisms suitable as neoepitope peptides and T-cells targets by mining mutation databases containing large repositories of potential neoepitopes that may be used to expand T-cells for immunotherapy.

The COSMIC database contains mutation data for 4754 transitional cell carcinomas, both whole exome sequencing and hotspot-analyses. The inventors focused on urinary bladder cancer (UBC) and selected the 15 of the most prevalent mutations resulting in a single amino acid mutation, and specifically a substitution mutation, thus qualifying as a neoepitope. The inventors also focused on polymorphisms in genes known to be associated with tumour pathogenesis such as kinases, growth factor receptors and cell cycle proteins. The chosen 15 mutations alone cover 73% of bladder cancer mutations found in COSMIC. The neoepitope peptides identified from the COSMIC database are referred to in this Example as “predicted neoepitope peptides”.

As an alternative, whole genome sequencing of a tumour from a patient with UBC is carried out in order to identify additional polymorphisms and new targets for immunotherapy. In RNA sequencing of tumours can be carried out to verify the presence of transcripts carrying the polymorphisms. Multiple reaction monitoring (MRM) mass spectrometry transitions can be used to quickly scan patients for expression of the most common neoepitopes at the protein level, tailoring the neoepitope peptides used for individual immunotherapy. The neoepitope peptides identified using this method are referred to in this Example as a “personalised neoepitopes”.

Neoantigenic constructs were designed as a 21 amino acid peptide with a somatic mutated amino acid located at the central 1 amino acid of the sequences (i.e. at amino acid position 11). To design neoantigenic constructs, three neoepitope peptides were linked with two VVR-spacers, since the VVR motif is cleaved by Cathepsin S in lysosomes, after translation, as a step in human leukocyte antigen presentation.

(iii) T-Cell Activation and Expansion by Neoepitopes

Nine neoepitopes were identified bioinformatically, as described above. The predicted neoepitope peptides were based on genes that harbour reported urinary bladder cancer associated mutations such as FGFR3 and p53. In a pilot experiment using a neoantigenic construct comprising 3 neoepitope peptides, the inventors were able to identify IFN-γ producing T-cells from blood of a patient with urinary bladder cancer by FluoroSpot, demonstrating the validity of the neoepitope peptide approach.

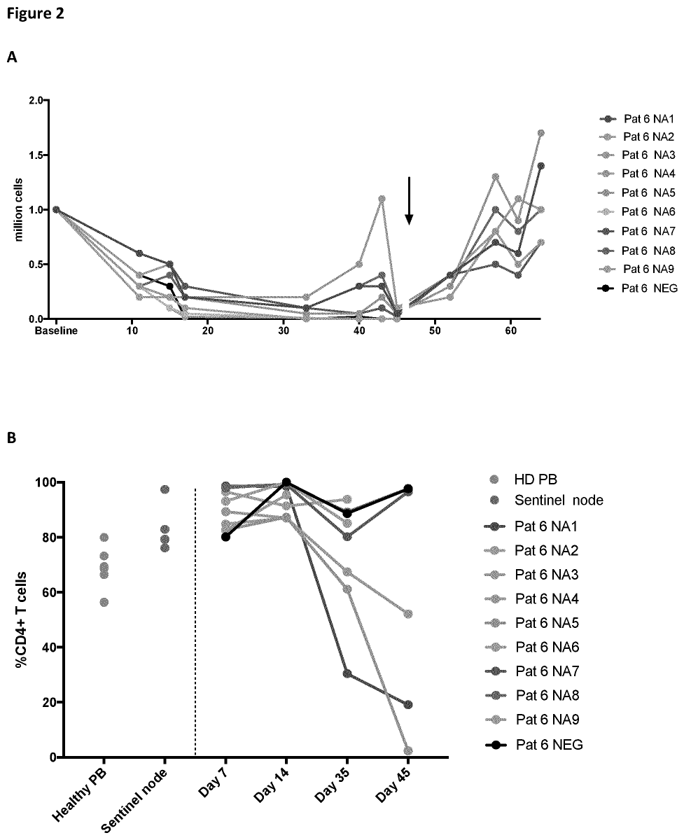

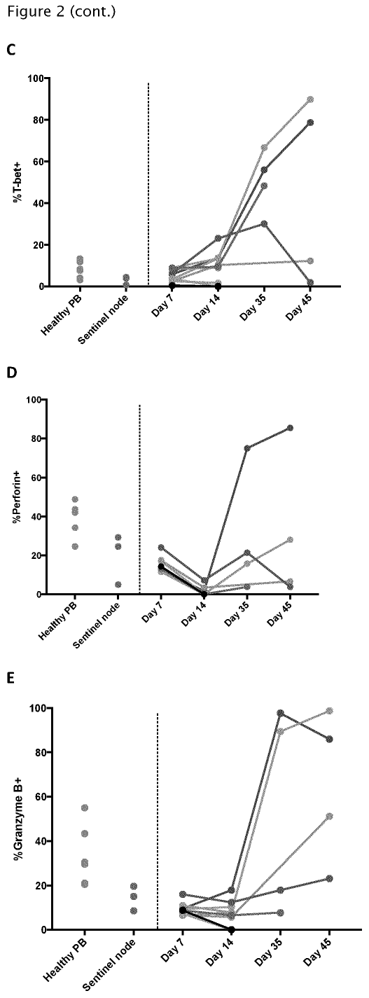

For the same patient having urinary bladder cancer, activation of T-cells was performed using the predicted neoepitope peptides NA1-9 (SEQ ID NOs: 4-12, see Table 4 below). Proliferation was seen in response to NA 1, 3, 5, 7 and 8 (SEQ ID NOs: 4, 6, 8, 10 and 11). FIG. 2A displays the number of cells in the PBMC culture over time (PB=Peripheral blood) after incubation with APCs that were contacted with phagocytosable particles comprising predicted neoepitope peptides (NA1-9) attached to polystyrene particles. The arrow in FIG. 2A indicates the time of re-stimulation (i.e. the time when the T-cell sample was contacted with a second batch of APCs contacted with the phagocytosable particles comprising the predicted neoepitope peptides (NA1-9) under conditions allowing specific activation of anticancer T-cells in response to a neoepitope presented by the APC). FIG. 2B shows the % CD4+/total T-cells. FIG. 2C shows the T-bet expression in CD4, and FIGS. 2D and E, show the expression of Granzyme B and Perforin in CD8+ T-cells.

The expanded T-cells express the transcription factor T-bet and high levels of the effector molecules Perforin and Granzyme B (GZB).

The method of the invention has also been used on cells from a patient with disseminated colon cancer from whom sequenced tumour data were available. The patient displayed two polymorphisms in p53, one known and one novel. The patient also displayed a mutation in PIK3CA. A personalised neoantigenic construct comprising three neoantigenic construct comprising thee neoepitope peptides and having an amino acid sequence according to SEQ ID NO: 3 was designed, expressed and purified, based on the specific mutations in the tumour data for the patient, which allowed the identification of personalised neoepitopes. Phagocytosable particles were prepared by coupling the neoantigenic construct to polystyrene particles following the protocols outlined in Examples 1 and 2. The phagocytosable particles comprising the personalised neoantigenic construct (SEQ ID NO: 3) were used for expanding cells, resulting in a neoepitope specific response. Peripheral blood mononuclear cells (PBMCs) were used for culture. FIG. 3 shows the percent among T-cells (small squares) and total number (large squares) of CD4+ T-cells, as well as proliferating CD4+ cells (circles) in the T-sample before and during the expansion with the phagocytosable particles comprising a polystyrene particle core and the personalised neoantigenic construct tightly associated to the core.

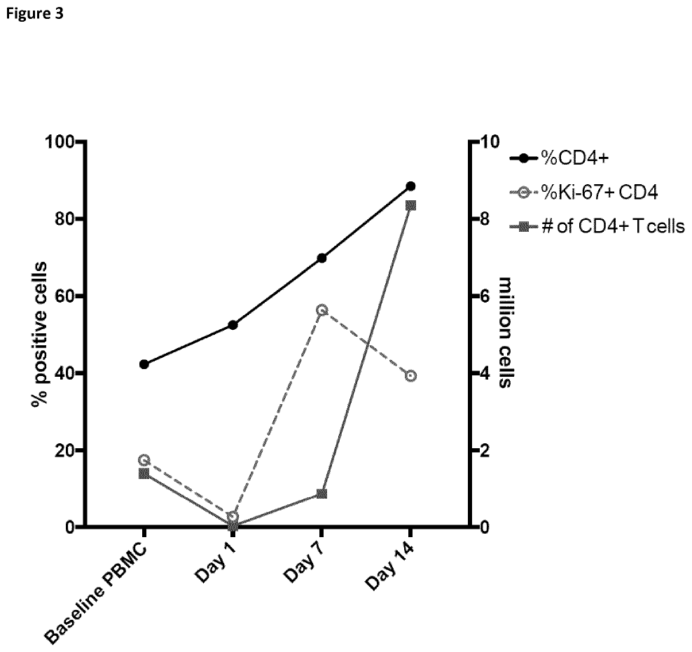

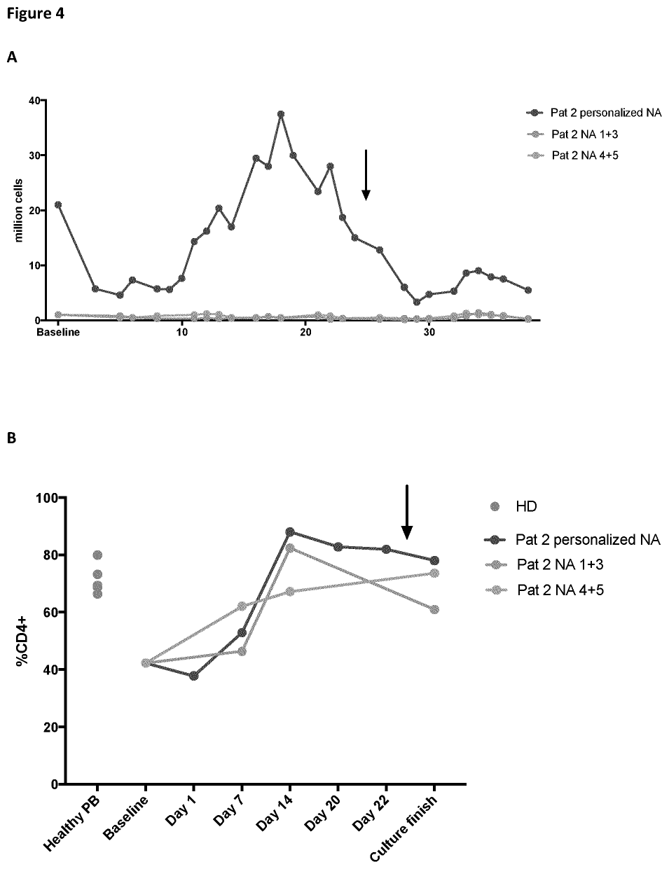

FIG. 4A shows the number of cells in the PBMC culture over time (days). The top line (Pat 2 personalised NA) shows for the number of cells in the PBMC culture after incubation that were contacted with the the phagocytosable particles comprising the personalised neoantigenic construct (SEQ ID NO: 3). The two lines at the bottom of the figure (Pat2 NA 1+3 and Pat 2 NA 4+5) display the number of cells in parallel PBMC cultures that were contacted with the phagocytosable particles of NA1 and NA3 or NA4 and NA5. FIG. 4B shows the % CD4+/total T-cells. The top line in FIG. 4B for the time points of 14 days and thereafter are for the Pat 2 personalised NA experiment. The arrows in FIGS. 4A and 4B indicate the time of re-stimulation (i.e. the time when the T-cell sample was contacted with a second batch of APCs contacted with phagocytosable particles under conditions allowing specific activation of anticancer T-cells in response to neoepitopes presented by APCs). FIG. 4C visualizes an analysis performed with the Barnes-Hut Stochastic Neighbor Embedding (BH-SNE) algorithm for CD4+ T-cells, where all cells in the samples are clustered on a 2-dimentional map according to the similarity in expression intensity according to a set of chosen markers, here CD28, CD57, T-bet, GATA-3, Perforin, Granzyme B (GZB), Ki-67 and PD-1.

The expression of the proliferation marker Ki67 and the number of T-cells increased in the expansion, and the expression of important markers for anti-tumour activity, such as T-bet, Perforin and Granzyme B, increased on both CD4+ and CD8+ cells over the 14-day culture period. The percentage of CD8+ T-cells decreased to around 10% of CD4+ T-cells, but the total number of CD8+ cells increased.

The whole process from receiving sequence data to analysis of expanded cells can be completed in 4-5 weeks.

These results show that there was a neoepitope specific T-cell response. Thus, the inventors have demonstrated that predicted and personalised neoepitope peptides can be designed and used for T-cell activation and expansion.

The method of expanding T-cells of this Example, and the T-cells expanded using the expansion method of this Example, find utility in the uses and methods described herein for the treatment and prophylaxis of cancer.

TABLE 4

| SEQ ID NO: | Sequence |
|---|---|
| 1 (M272120) | PSFQEFVDWENVSPELNSTDQVVRHLLGRLAAIVG |
| | KQVVLGRKVVVVRHWNDLAVIPAGVVHNWDFEPR |
| 2 (M304748) | VELCPGNKYEMRRHGTTHSLVVVRDEVALVEGVQS |
| | LGFTYLRLKDVVRKAFLHWYTGEAMDEMEFTEAE |
| 3 | EAPRMPEAAPRVAPAPAAPTPVVRQSQHMTEVVRH |
| | CPHHERCSDSVVRCATYVNVNIRNIDKIYVRTG |
| 4 (NA1) | AQTYTLDVLERCPHRPILQAGLVVRSTRDPLSEITK |
| | QEKDFLWSHRVVRLVEADEAGSVCAGILSYGVGFGS |
| 5 (NA2) | AKAISTRDPLSKITEQEKDFLWVVRPYNYLSTDVGF |
| | CTLVCPLHNQVVRRQTYTLDVLECSPHRPILQAGGS |
| 6 (NA3) | AALLALWLCCATPAHALQCRDGVVRVKEGWLHKRGK |
| | YIKTWRPRYFVVREYFMKQMNDARHGGWTTKMDWGS |
| 7 (NA4) | ATEYKLVVVGAVGVGKSALTIQVVREEELVEADEAC |
| | SVYAGILSYGVVRCACPGRDRRTKEENLRKKGEPGS |
| 8 (NA5) | ATEYKLVVVGADGVGKSALTIQVVRFEVRVCACPGT |
| | DRRTEEENLRVVRCLLDILDTAGREEYSAMRDQYGS |
| 9 (NA6) | AMASAAAAEAEKGSPVVVGLLVVGNIIILLSGLSLF |
| | AETIWVTADQYRGS |
| 10 (NA7) | ACFQGLLIFGNVIIGCCGIALTAECIFFVSDQHSLYPL |
| | LEATDNDDIYGAAWIGIFVGICLFCLSVLGIVGIMKGS |
| 11 (NA8) | ATLPLILILLALLSPGAADFNISSLSGLLSPALTESLL |
| | VALPPCHLTGGNATLMVRGS |
| 12 (NA9) | AFGSAVNLQPQLASVTFATNNPTLTTVALEKPLCMFDS |
| | KEALTGTHEVYLYVLVDSAISRGS |

### Example 6: Tumour Mouse Model Assessing Tumor Growth Following Vaccination with Phagocytosable Particles Coupled to MC38 Colorectal Tumor-Specific Neoantigens

Materials and Method:

A phagocytosable particle composition comprising six different phagocytosable particles groups was used in this study (herein referred to as the “MC38 particle composition”). Each particle group comprised a polystyrene particle core (Sera-Mag SpeedBeads (hydrophilic) Carboxylate-Modified Magnetic particles, GE Healthcare) coupled to one of the following types of MC38 neoantigen constructs 1) SEQ ID NO: 13, 2) SEQ ID NO: 14, 3) SEQ ID NO: 15, 4) SEQ ID NO: 16, 5) SEQ ID NO: 17 or 6) SEQ ID NO: 18 (see Table 5).

TABLE 4

| SEQ ID NO | Sequence |
|---|---|
| 13 (MC38 #1) | MGSSHHHHHHSSGSLAEAKVLANRELDKYGVSDYHKNLINNAKTVEGVKDLQAQ |
| | VVESAKKARISEATDGLSDFLKSQTPAEDTVKSIELAEAKVLANRELDKYGVSD |
| | YYKNLINNAKTVEGVKALIDEILAALPGGSAYEGDGGDASRVLEDSNISYGSGG |
| | SREPVAATWEASWSEGSKSLDSGGSKATGSPTPRINWLKGGRPLSLGGSTSWLM |
| | LPDGINVEVIVVNQVNGGSYILLVGYPPFCDEDQHKLYQQGGSDGNNNLEDDSI |
| | VSEDLDVDWSSG |
| 14 (MC38 #2) | MGSSHHHHHHSSGSLAEAKVLANRELDKYGVSDYHKNLINNAKTVEGVKDLQAQVVESAK |
| | KARISEATDGLSDFLKSQTPAEDTVKSIELAEAKVLANRELDKYGVSDYYKNLINNAKTV |
| | EGVKALIDEILAALPGGSASQGELIHPKAFPLIVGAQLIHGGSKRKEQEAQEEKRRKQRE |
| | AQAWGGSMGPGAGRPWPSPNSANSIPYSGGSDRVPNVRVLLTKTLRQTLLEKGGSISLAF |
| | FEAASIMRQVSHKHIVGGSSNYQLGELVKLENYPDVIRLISG |
| 15 (MC38 #3) | MGSSHHHHHHSSGSLAEAKVLANRELDKYGVSDYHKNLINNAKTVEGVKDLQAQVVESAK |
| | KARISEATDGLSDFLKSQTPAEDTVKSIELAEAKVLANRELDKYGVSDYYKNLINNAKTV |
| | EGVKALIDEILAALPGGSAKVHAVFLDGVKVTLNWHLSSSGGSMMLGPEGGESYVVKLRG |
| | LPWGGSKGTIVAQVDSIESFQEFCSTSGGSKYMCNSSCMGVMNRRPILTIIGGSEEKQAA |
| | KKRKLEESVEQKRSKGGSTWSLASITYWRPTCANTVSDNSG |
| 16 (MC38 #4) | MGSSHHHHHHSSGSLAEAKVLANRELDKYGVSDYHKNLINNAKTVEGVKDLQAQVVESAK |
| | KARISEATDGLSDFLKSQTPAEDTVKSIELAEAKVLANRELDKYGVSDYYKNLINNAKTV |
| | EGVKALIDEILAALPGGSARKRPYSSFSNCKDHREWDHYRGGSRNVMCKKDSPLRTTTIV |
| | PPVEGGSHNCLSDPADHRRLTEHVAKAFGGSSAGGWGTEILWSTFAFKASRQGGSPPA |
| | DFTQPAASAAAAAVAAAAGGSQNAGGSVMIQLVNGSLAVSRASG |
| 17 (MC38 #5) | MGSSHHHHHHSSGSLAEAKVLANRELDKYGVSDYHKNLINNAKTVEGVKDLQAQVVESAK |
| | KARISEATDGLSDFLKSQTPAEDTVKSIELAEAKVLANRELDKYGVSDYYKNLINNAKTV |
| | EGVKALIDEILAALPGGSAGEDNRPGMRGCHQMVIDVQTEGGSGQIQEESEGARFKAPP |
| | DSTVSGGSSVAAAAAAAVSVVESMVTATEGGSVSHKHIVYLYVVCVRDVENIMGGSEYL |
| | KLLHSFVYSVGFVTSPFSGGSDAVASFADVGFVATEEGECSISG |
| 18 (MC38 #6) | MGSSHHHHHHSSGSLAEAKVLANRELDKYGVSDYHKNLINNAKTVEGVKDLQAQVVESAK |
| | KARISEATDGLSDFLKSQTPAEDTVKSIELAEAKVLANRELDKYGVSDYYKNLINNAKTV |
| | EGVKALIDEILAALPGGSAVVDHRPKALPVGGFIEEEKDEGGSKQDEYHMVHLLCASRSP |
| | PSSPGGSGDTLEEAFEQSAMAMFGYMTDGGSISMSSSKLLLSAKALSTDPASGGSRDLG |
| | DEYGWKHVHGDVERPSSGGSRVSLSHACKNTVKTDAPPEALSG |

Each MC38 neoantigenic construct includes six different 20-23 amino acid neoantigen peptide sequences derived from the MC38 colon cancer cell line. The MC38 neoantigenic constructs were recombinantly expressed using E. coli and purified using column chromatography.

The MC38 particles were prepared following the protocol outlined in Example 1. Once made, the six groups of phagocytosable particle were mixed, and then sterilised using the protocol described in Example 2. A stock solution of the MC38 particle composition was prepared at a concentration of 10 million particles/μl in PBS.

Test mice (n=5) received a first and a second dose of the MC38-particle composition. Each dose had a total volume of 10 μl (9.5 μl of MC38 particle composition stock solution and 0.5 μl of 0.05 nmol CpG oligodeoxynucleotide (ODN 1668, Enzo) as an adjuvant). Control mice received no treatment (n=5, non-vaccinated group).

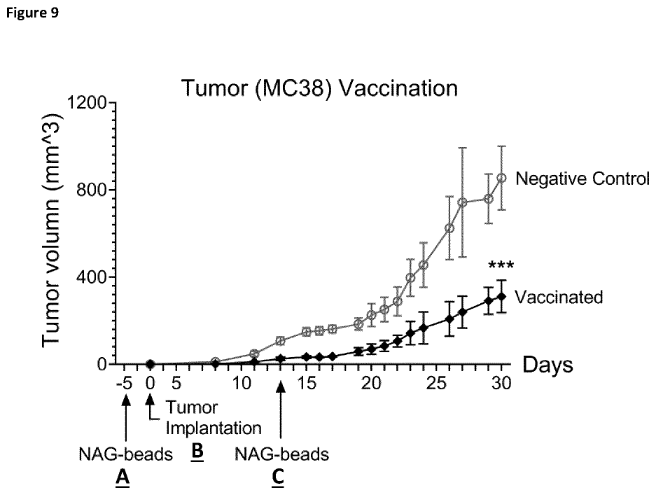

The first dose of the phagocytosable particles (MC38-particle composition) was injected five days before transplanting the MC38 tumour cells (time point A in FIG. 9). The MC38 tumour cells were transplanted on Day 0 (time point B in FIG. 9). Transplantation was achieved by injecting 10⁶ MC38 tumour cells into each mouse. Thirteen days after transplantation, the mice were injected with a second dose (time point C in FIG. 9) of the same type of phagocytosable particles they received as a first dose (i.e. MC38-particles). The tumour volume in each mouse was measured over the course of the study (see FIG. 9).

Results

The results are shown in FIG. 9. As can be seen from FIG. 9, tumour growth was notably reduced in the mice that received the MC38-particles (“vaccinated” mice) compared to the non-vaccinated mice (“negative control” mice). These results indicate that the MC38-particles are effective at inducing an anti-cancer immune response that inhibits tumour growth in a MC38 colorectal tumor mouse model.

### Example 7: Toxicity and Biodistribution Study

To assess the maximum tolerated dose of the phagocytosable particles, the toxicity and biodistribution profile of the core of the phagocytosable particles are assessed using a rat model.

Materials and Method:

Particles used in study: Sera-Mag SpeedBead Carboxylate-Modified Magnetic Particles (Hydrophilic). These particles are supplied by GE Healthcare Life Sciences (Particle Lot/Batch Number GE: 16807675, concentration: 2.3% solids (g/100 g) (corresponding to 10 mg Fe/mL) in Dulbecco's phosphate buffered saline, pH 7.1). The particles are stored prior to use at 2-8° C.

Animal details: Rats, Wistar. On arrival to the study location, the rats have an approximate weight of 250 g. Rats are acclimatised for a minimum of 5 days prior to the start of the study. The rats are kept in individually ventilated cages (type IVC 4) at +22° C.±3° C., a humidity of 50%±20% and with 12-hour light/12-hour dark cycles. Food and water are available ad libitum. There are three rats per cage.

Study 1):

Five female Wistar rats are used in the pilot study. Rat #1 is subjected to intravenous (IV) injection with the maximum feasible concentration of particle (concentration equivalent to 50 mg/kg iron at 5 mL/kg), after which the rat is placed in a computed tomography (CT) camera for acquisition of an image. Intravenous injection is performed as slowly as possible. If a toxic response is evident, particles are delivered to the remaining rats by slow infusion over a period of 20 minute (max 20 mL/kg). The particle concentration delivered to the rats is titrated until a maximum tolerated dose is identified. A rat that has not received a dose of particles is used as a negative control and for acquisition of a background CT scan.

Following IV injection/infusion of the particles, the rats are anaesthetised with isoflurane and ophthalmic ointment is placed on the eyes to prevent dehydration. Rats are then placed on the heated bed of a CT instrument, where inhalation anaesthesia is maintained. Vital parameters (pulse and respiration) are monitored during the course of the experiment using a respiration sensor and rectal thermometer. Rats are inserted into the CT camera, where a picture is acquired over the course of about 20 minutes. Health status following IV injection of particles is documented to assess possible toxic reactions, and a whole-body CT image is used to assess particle visibility and to determine the location of particles following injection. Following acquisition of a CT image, the rats are euthanised.

Study 2):

A further study is performed using twelve rats to identify the rate of particle elimination. All rats receive an IV injection of the particles at a dose identified in the pilot study. Immediately after administration of the particles, the rats are placed in a CT camera. The rats are thereafter monitored in the CT camera at 24 h, 3 days, 7 days, 14 days and 1 month after administration. At each time point, two rats are euthanised for excision of organs for histopathological analysis. The rats are monitored for changes in health status and body weights 24 h, 3 days and 7 days after administered and once weekly thereafter.

### Example 7: Exemplification of Phagocytosable Particle Sterilisation Protocol Using Bacillus subtilis

This experiment was performed under sterile conditions in a laminar air flow (LAF) safety cabinet. Phagocytosable particles comprising a core (Sera-Mag SpeedBeads Carboxylate-Modified magnetic particles, GE Healthcare) attached to a neoantigen construct were washed four times with high concentration alkaline solution (2M to 5M NaOH). After the first wash, the phagocytosable particles were transferred to a new sterile tube, the supernatant was removed, and a second volume of alkaline solution was added. Phagocytosable particles were sonicated for 10 minutes in a sonication bath, and then incubated for 30 minutes with end-over-end rotation in the same alkali solution. This process was repeated a further two times, followed by four washes with sterile Dulbecco-modifies PBS.

Effectiveness of the NaOH treatment protocol was evaluated by spiking phagocytosable particles (Sera-Mag SpeedBeads Carboxylate-Modified magnetic particles, without attached neoantigen) with a high load (>1.2 CFU) of Bacillus subtilis subsp. spizizenii (ATCC® 6633™ Epower 106 CFU), followed by the above described NaOH treatment protocol. After NaOH treatment, both full bead suspension and supernatant from non-treated (positive control) vs 5M or 2M NaOH treated samples were plated on nutrient agar in the absence of antibiotics, and incubated at 37° C. overnight (>16 hours).

Results

Both 5M and 2M NaOH treatment effectively abolished bacterial growth, whereas colonies of Bacillus subtilis subsp. spizizenii were abundantly growing in the absence of washes. In conclusion, both the 2M and 5 M NaOH treatments were highly effective at removing an artificially high bioburden load from the phagocytosable particles.

1. A phagocytosable particle for use in the treatment or prophylaxis of cancer in a subject, wherein the phagocytosable particle comprises a core and a neoantigenic construct tightly associated to the core, and wherein the neoantigenic construct comprises a neoepitope peptide having an amino acid sequence corresponding to an amino acid sequence of a part of a protein or peptide known or suspected to be expressed by a cancer cell in the subject, wherein the part of the protein or peptide has at least one somatic mutated amino acid.

2. A phagocytosable particle for use according to claim 1, wherein the neoantigenic construct comprises two or more covalently linked neoepitope peptides.

3. A phagocytosable particle for use according claim 2, wherein the neoantigenic construct comprises three or more covalently linked neoepitope peptides; for example three, four or five covalently linked neoepitope peptides.

4. A phagocytosable particle for use according to claim 2 or 3, wherein the covalently linked neoepitope peptides are covalently linked via a spacer moiety.

5. A phagocytosable particle for use according to claim 4, wherein the spacer moiety is a sequence of 1 to 15 amino acids, preferably 1 to 5 amino acids, and more preferably comprising the amino acid sequence VVR and/or the amino acid sequence GGS.

6. A phagocytosable particle for use according to any one of claims 2 to 5, wherein each of the covalently linked neoepitope peptide is 3 to 25 amino acids in length.

7. A phagocytosable particle for use according to any preceding claim, wherein the neoantigenic construct is covalently attached to the core.

8. A phagocytosable particle for use according to any preceding claim, wherein the phagocytosable particle comprises two or more different neoantigenic constructs tightly associated to the core, for example two, three, four or five neoantigenic constructs tightly associated to the core.

9. A phagocytosable particle for use according to claim 8, wherein each of the different neoantigen constructs comprise different neoepitope peptide sequences or a different combination of neoepitope peptides.

10. A phagocytosable particle for use according to any preceding claim, wherein the phagocytosable particle has a largest dimension of less than 5.6 μm, preferably less than 4 μm, more preferably less than 3 μm, even more preferably from 0.5 to 2 μm, or most preferably about 1 μm.

11. A phagocytosable particle for use according to any preceding claim, wherein the core is paramagnetic or superparamagnetic.

12. A phagocytosable particle for use according to any preceding claim, wherein the core comprises a polymer, preferably polystyrene.

13. A phagocytosable particle for use according to any preceding claim, wherein the phagocytosable particle is administered with an adjuvant, or comprises an adjuvant tightly associated to the core (e.g. IL-2, IL-15, IL-17 and IL-4).

14. An injectable pharmaceutical composition comprising a phagocytosable particle comprising a core and a neoantigenic construct tightly associated to the core, wherein the neoantigenic construct comprises a neoepitope peptide having an amino acid sequence corresponding to an amino acid sequence of a part of a protein or peptide known or suspected to be expressed by a cancer cell in the subject, wherein the part of the protein or peptide has at least one somatic mutated amino acid.

15. An injectable pharmaceutical composition according to claim 14, wherein the phagocytosable particle is defined in any one of claims 2 to 13.

16. An injectable pharmaceutical composition according to claim 14 or 15, for use in the treatment or prophylaxis of cancer in a subject.

17. A phagocytosable particle for use according to claims 1 to 13, or an injectable pharmaceutical composition for use according to claim 16, wherein the cancer is a solid cancer, for example a cancer selected from breast cancer, colon cancer, liver cancer, lung cancer (non-small cell and small cell), lung carcinoid tumour, pancreatic cancer, prostate cancer, ovarian cancer and urinary bladder cancer.

18. A method of treating or preventing cancer in a subject which comprises administering to the subject a phagocytosable particle, wherein the phagocytosable particle comprising a core and a neoantigenic construct tightly associated to the core, wherein the neoantigenic construct comprises a neoepitope peptide having an amino acid sequence corresponding to an amino acid sequence of a part of a protein or peptide known or suspected to be expressed by a cancer cell in the subject, wherein the part of the protein or peptide has at least one somatic mutated amino acid

19. A phagocytosable particle for use according to any one of claims 1 to 13, or an injectable pharmaceutical composition for use according to claim 16, or a method according to claim 18, wherein the treatment or prophylaxis of cancer further comprises the step of:
administering one or more subsequent doses to the subject of the phagocytosable particle or injectable pharmaceutical composition, wherein the subject is one whom has previously been administered a dose of the phagocytosable particle or injectable pharmaceutical composition sufficient to elicit an immune response towards a cancer cell in the subject.

20. A phagocytosable particle for use according to any one of claims 1 to 13, 17 or 19, or an injectable pharmaceutical composition for use according to claims 16, 17 or 19, wherein the treatment or prophylaxis of the cancer in the subject further comprises the steps of:
a) harvesting APCs and anticancer T-cells from the subject after the administration of the phagocytosable particle to the subject;
b) expanding the anticancer T-cells harvested from the subject; and
c) administering a therapeutic dose of the expanded anticancer T-cells to the subject.

21. A phagocytosable particle or an injectable pharmaceutical composition for use according to claim 20, wherein the APCs and anticancer T-cells are harvested from a PBMC sample derived from the subject; for example from the APCs and anticancer T-cells are harvested from the same PBMC sample, or the APCs and anticancer T-cells are harvested from different PBMC samples.

22. A phagocytosable particle or an injectable pharmaceutical composition for use according to claim 20 or 21, wherein the anticancer T-cell activation and expansion step b) comprises the steps of:
i. providing a phagocytosable particle phagocytosable particle comprising a core and a neoantigenic construct tightly associated to the core, wherein the neoantigenic construct comprises a neoepitope peptide having an amino acid sequence corresponding to an amino acid sequence of a part of a protein or peptide known or suspected to be expressed by a cancer cell in the subject, wherein the part of the protein or peptide has at least one somatic mutated amino acid;
ii. providing an APC;
iii. contacting the phagocytosable particle with the APC in vitro, and under conditions allowing phagocytosis of the phagocytosable particle by the APC;
iv. providing anticancer T-cells harvested from the subject;
v. contacting the anticancer T-cells with the APC from step iii) in vitro, and under conditions allowing specific activation of anticancer T-cells in response to neoepitopes presented by the APC.
